(19) United States
(12) Patent Application Publication
(10) Pub. No.: US 2025/0163020 A1
(43) Pub. Date: May 22, 2025

(54) Chiral Synthesis of Nornicotine and Nicotine

(71) Applicants: SIEGFRIED AG, Zofingen (CH); CONTRAF-NICOTEX-TOBACCO GMBH, Heilbronn (DE)

(72) Inventors: Karolína STRAKOVÁ, Zofingen (CH); Dominik Simon MÜLLER, Frenkendorf (CH); Cornelis Hendrik VAN VEGTEN, Brugg (CH); Florian Daniel ZELLER, Trimbach (CH); Thomas DEBNAR, Kriens (CH)

(73) Assignees: SIEGFRIED AG, Zofingen (CH); CONTRAF-NICOTEX-TOBACCO GMBH, Heilbronn (DE)

(21) Appl. No.: 19/029,517

(22) Filed: Jan. 17, 2025

Related U.S. Application Data
(63) Continuation-in-part of application No. PCT/EP2023/073883, filed on Aug. 31, 2023.

(30) Foreign Application Priority Data
Aug. 31, 2022 (EP) 22193139.7

(51) Int. Cl. C07D 401/04

(52) U.S. Cl. CPC C07D 401/04

(57) ABSTRACT

The present invention relates to a method of producing nornicotine through enantioselective hydrosilylation of myosmine and a composition produced thereby, as well as a composition comprising nornicotine and a catalyst, wherein the catalyst is an organic chiral Lewis base, and a catalyst. The present invention also relates to a method of producing nicotine or a salt thereof from myosmine through enantioselective hydrosilylation of myosmine to nornicotine and further reacting the nornicotine to nicotine or a salt thereof, and a composition produced thereby, as well as a composition comprising nicotine, or a salt thereof, and a catalyst, wherein the catalyst.

### FIELD OF THE INVENTION

The present invention relates to a method of producing nornicotine through enantioselective hydrosilylation of myosmine and a composition produced thereby, as well as a composition comprising nornicotine and a catalyst, wherein the catalyst is an organic chiral Lewis base. The present invention also relates to a method of producing nicotine or a salt thereof from myosmine through enantioselective hydrosilylation of myosmine to nornicotine and further reacting the nornicotine to nicotine or a salt thereof, and a composition produced thereby, as well as a composition comprising nicotine, or a salt thereof, and a catalyst, wherein the catalyst is an organic chiral Lewis base. The present invention relates particularly to the enantioselective preparation of nicotine from myosmine without the need of extensive purification steps and metal catalysts.

### BACKGROUND OF THE INVENTION

Nicotine is a naturally occurring alkaloid used in various applications. Especially (S)-nicotine is used as active pharmaceutical ingredient to treat nicotine abuse and nicotine dependency. Success has further been reported when treating Tourette's syndrome, Alzheimer's disease, schizophrenia and other diseases related to disorders of the nervous system. Common ways of administration are gums, creams, transdermal patches, tablets, nasal sprays, and electric cigarettes. Considerable quantities of nicotine are also used in agriculture as plant protectant or pesticide against aphids. Natural nicotine is extracted from tobacco plants, a process that requires efficient purification steps to remove undesired, harmful impurities. The increasing demand of nicotine creates a need to offer ecological and economical ways to prepare synthetic nicotine in very pure form.

### STATE OF THE ART

Nicotine ((S)-3-(1-methylpyrrolidin-2-yl)pyridine) and its enantiomers have been prepared for many years by various methods. Known syntheses are usually expensive and use agents that are problematic or even toxic for the environment. In order to obtain the desired (S) enantiomer of nicotine, it is often the case that first, the racemic mixture of (R) and (S) nicotine is produced and in a second step, the enantiomers are separated.

Pictet A. reported in 1904 already a synthesis for nicotine, including the use of tartaric acid to separate the enantiomers (Berichte der deutschen chemischen Gesellschaft, vol. 37, 1904, pages 1225-1235). Tartaric acid has then been used afterwards for decades (see for example: Aceto M. D., et al. (J. Med. Chem., 1979, vol. 22, 17 4-177)). Katsuyama A. et al. reported a way of synthesizing nicotine using potassium tert-butanolate for the racemization of nicotine to prepare the starting material for further separation of the enantiomers (Bull. Spec. CORESTA Symposium, Winston-Salem, 1982, p. 15, S05, ISSN 0525-6240). Further, EP 4 487 172 discloses a synthesis route over 5 different steps, offering a net yield of 37.7%. WO 2017/117575 discloses a synthesis of nicotine using potassium hydride (KH) or sodium hydride (NaH) as strong base in tetrahydrofuran (THF) as solvent to obtain niciotinoyl-1-vinylpyrrolidin-2-one. Yield of (R/S)-nicotine is about 31%. Similar procedures as well as procedures for enantiomeric separation of the nicotine have been disclosed by Wang J. et al. (Wang J. et al., E. J Med. Chem., 2017, vol. 130, 15-25), by Desai D. et al. (Desai D. et al., J. Labeled Compd. Radiopharm, 2008, vol. 51, 226-230), Aceto M. D. et al., (Aceto M. D. et al., J. Med. Chem., 1979, vol. 2, 174-177) or by Bowman E. R. et al. (Bowman E. R. et al., Synthetic Comm., 1982, vol. 12, 11, 871-879). In US 2016/0326134 a synthesis comprising the condensation of 1-methylpyrrolidin-2-one and methyl nicotinate in the presence of a strong base (as K tert-butoxide) at reflux to the intermediate potassium 1-methyl-3-nicotinoyl-4,5-dihydro-1H-pyrrol-2-olate is described, which then can be converted into the racemic mixture of R/S nicotine. Di-para-toluoyl-L-tartaric acid serves as resolution agent. EP 2 484 673 (U.S. Pat. No. 8,378,111) relies on well-known routes of synthesis and discloses D-DBTA (D-dibenzoyl ester of tartaric acid) as agent to separate the enantiomers. WO 2016/065209 (EP 3 209 653, U.S. Pat. No. 9,556,142) discloses a preparative way comprising three steps to the intermediate myosmine, including the condensation of N-vinylegous-pyrrolidinone and nicotinate ester in the presence of a metal hydride. WO 2019/121644 (EP 3 728 215) teaches a one-pot synthesis of nicotine starting from ethyl nicotinate and N-vinylpyrrolidone. The method further comprises the steps of the reduction of myosmine to nornicotine and the methylation of the nornicotine to nicotine.

However, it is desirable to establish a synthesis route which opens the possibility of the enantioselective synthesis of (S)-nicotine or (R)-nicotine. This would render the resolution step redundant and would lead to a more efficient and ecological method for the production of enantiopure nicotine. In this regard, a possible strategy is the stereoselective reduction of 3-myosmine to (S)- or (R)-nornicotine, wherein the nornicotine is then further reacted to nicotine. WO 2020/098978 (EP 3 653 617, US 2020/0157589) discloses a process for the production of (S)-nicotine from myosmine by enzymatic reduction and reductive methylation. Buchwald S. et al. described the enantioselective catalytic hydrosilylation of myosmine to nornicotine using the titanium catalyst (S,S)-(EBTHI)TiF₂ (Buchwald S. et al., J. Am. Chem. Soc., 1996, vol. 118, 6784-6785). Through this method, it is possible to synthesize (S)- and (R)-nornicotine in high yields and enantiomeric excess (ee) with low catalyst loadings. The enantioselective synthesis of (S)-nicotine from myosmine derivates through an Ir-catalyzed hydrogenation was reported by Zhou et al. (Zhou et al., J. Am. Chem. Soc., 2015, vol. 137, 90-93). Fujieda et al. discloses the Ir-catalyzed assymetric hydrogenation of a pyrroline derivate (Fujieda et al., Synth. Commun., 2019, vol. 49, 814-822).

The disadvantage of the routes described above is the use of expensive metal-organic catalysts or enzymes. In the case of metal catalysts, the metal has to be separated from the nicotine (or nornicotine), especially in view of pharmaceutical and agritechnical applications of nicotine.

Lundrigan et al. shows the enantioselective imine reduction with boranes catalyzed by a diazaphosphenium triflate (Lundrigan et al., J. Am. Chem. Soc., 2019, vol. 141, 14083-14088).

However, boranes are expensive and produce boric acid upon hydration, which is a substance on the Candidate List of Substances of Very High Concern of ECHA (European Chemicals Agency). The stereoselective hydrosilylation of acylic imines in the presence of an organocatalyst is reported by Benaglia et al. (Benaglia et al., ChemCatChem, 2017, vol. 9, 941-945). Through this method, a variety of chiral amines were produced with moderate to high yields and enantiomeric excess (ee) at mild reaction conditions. Y. C. Xiao et al, Angew.

Chem. Int. Ed., 2011, vol. 50, 10661-10664 discloses the asymmetric hydrosilylation of indoles using combined Lewis Base and Brønsted acid activation. X. Li et al. Org. Biomol. Chem., 2017, Vol. 15, 2422-2435 provides mechanistic investigations of the asymmetric hydrosilylation of ketimines with trichlorosilane.

Thus, an object of the present invention is the provision of a method for the enantioselective production of nornicotine and nicotine from myosime, particularly at low temperatures, without the application of metal catalysts, biocatalysts, boranes and/or extensive racemate splitting, wherein the catalyst applied is easy to produce with readily accessible starting materials.

### SUMMARY OF THE INVENTION

The present invention offers a novel method for the enantioselective synthesis of nornicotine and nicotine. Inventors found a method of such syntheses without the application of a metal catalyst. This reduces the amount of possible metal impurities in the nornicotine as well as in the end product nicotine, which is particularly useful when it is to be used as active pharmaceutical ingredient, being subject to strict purity regulations. Furthermore, metals used as or in catalysts for hydrogenation reactions are often expensive precious metals such as iridium, platinum or palladium. Superseding the need of a metal catalyst thus leads to cost reduction and to a more ecological way of synthesizing nicotine. Also, the present invention particularly offers an enantioselective one-pot synthesis of nicotine from myosmine, which renders the resolution step redundant.

In a first aspect, the present invention relates to a method of producing nornicotine from myosmine as defined in claim 1. The invention also relates in another aspect to a composition which is produced by said method, particularly wherein the catalyst is not removed from the reaction mixture.

In a further aspect, the present invention relates to a composition as defined in claim 9.

In an even further aspect, the present invention relates to a method of producing nicotine or a salt thereof from myosmine as defined in claim 11. The invention also relates to a composition which is produced by said method, wherein the catalyst is not separated from the reaction mixture.

Additionally disclosed is a composition as defined in claim 13.

Further aspects and embodiments of the invention are disclosed in the dependent claims and can be taken from the following description, figures and examples, without being limited thereto.

### DETAILED DESCRIPTION OF THE PRESENT INVENTION

### Definitions

Unless defined otherwise, technical and scientific terms used herein have the same meaning as commonly understood by one of ordinary skill in the art to which this invention belongs.

Amounts within the present invention are given in wt. %, unless stated otherwise or clear from context.

Room temperature is 20 to 25° C., e.g. 22° C.

In structural formulae, Bn is a benzyl group, Bu is a n-butyl group, Et is an ethyl group, Me is a methyl group, Ph is a phenyl group, tBu is a tert-butyl group.

Pseudohalogen groups in the context of the present invention are not particularly limited. As is generally understood, pseudohalogen groups are polyatomic analogues of halogens.

According to certain embodiments, pseudohalogen groups are —SCN, —NCS, —CN, —N₃, —OCN, —NCO, or —SeCN.

An alkenyl group within the scope of the present invention comprises at least one double bond between two carbon atoms in a residue that is not aromatic and/or heteroaromatic, i.e. can comprise also more than one double bond. The position of the double bond is not particularly restricted and can be e.g. within an alkyl chain and/or at a terminal end.

An alkynyl group within the scope of the present invention comprises at least one triple bond between two carbon atoms in a residue that is not aromatic and/or heteroaromatic, i.e. can comprise also more than one triple bond. The position of the triple bond is not particularly restricted and can be e.g. within an alkyl chain and/or at a terminal end.

An aralkyl group within the scope of the present invention is a group wherein an alkyl residue is substituted with at least one aromatic residue. Within the scope of the invention, it is not excluded that the at least one aromatic residue is substituted with at least one further alkyl residue.

An alkylaryl group within the scope of the present invention is a group wherein an aromatic residue is substituted with at least one alkyl residue. Within the scope of the invention, it is not excluded that the at least one alkyl residue is substituted with at least one further aromatic residue.

A heterocycloalkyl group within the scope of the present invention is a cyclic alkyl group with at least one heteroatom in the alkyl chain, e.g. comprises O, N and/or S.

An alkyl heteroaromatic group within the scope of the present is a group wherein an alkyl residue is substituted with at least one heteroaromatic residue. Within the scope of the invention, it is not excluded that the at least one heteroaromatic residue is substituted with at least one further alkyl residue.

A heteroaromatic group with at least one alkyl residue within the scope of the present invention is a group wherein a heteroaromatic residue is substituted with at least one alkyl residue.

Within the scope of the invention, it is not excluded that the at least one alkyl residue is substituted with at least one further heteroaromatic and/or aromatic residue.

An alkyl (hetero)cycloalkyl group within the scope of the present invention is a group wherein a (hetero)cycloalkyl residue is substituted with at least one alkyl residue. Within the scope of the invention, it is not excluded that the at least one alkyl residue is substituted with at least one further (hetero)cycloalkyl residue.

A (hetero)cycloalkyl alkyl group within the scope of the present invention is a group wherein an alkyl residue is substituted with at least one (hetero)cycloalkyl residue. Within the scope of the invention, it is not excluded that the at least one (hetero)cycloalkyl residue is substituted with at least one further alkyl residue.

A carboxylic acid ester group within the scope of the present invention is a group wherein a carbon containing residue R′, like a linear, branched and/or cyclic alkyl residue and/or a (hetero)aromatic residue, is bonded via the functional group (CO)—O to form R′(CO)—O—, e.g. Me-(CO)—O or Et-(CO)—O.

An alkyl and/or (hetero)aryl oxycarbonyl group within the scope of the present invention is a residue wherein a linear, branched and/or cyclic alkyl and/or an aryl and/or an aryl residue R′ is bonded via the functional group O—(CO) to form R′O—(CO)—.

A carboxamide group within the scope of the present invention is either a group having the formula R′(CO)(NR″), wherein a carbon containing residue R′ like a linear, branched and/or cyclic alkyl residue and/or a (hetero)aromatic residue is bonded via the functional group (CO)NR″—, wherein R″ can be H, a carbon containing group, or a different element or group, or a group having the formula (NR″R′″)(CO)—, wherein R″ and R′″ can be H, a carbon containing group, or a different element or group, where an amine derived group (NR″R′″) is bonded via (CO).

The term “organic” within the scope of the present invention is meant in a strict sense, i.e. excluding metal-organic compounds. Further, an organic chiral Lewis base, within the scope of the invention, also does not include biological material such as enzymes.

In the context of the present invention, a Lewis base is not particularly limited. As is generally understood, a Lewis base is an electron donating compound which has a filled orbital containing an electron pair which is not involved in bonding.

In the context of the present invention, alkoxy groups with a linear, branched, cyclic and/or aromatic residue having 1 to 20 C atoms that can comprise one or more heteroatoms selected from O, N, S and P are not particularly restricted, as well as also carboxylic ester groups with a linear, branched, cyclic and/or aromatic residue having 1 to 20 C atoms that can comprise one or more heteroatoms selected from O, N, S and P, etc. In the residues, it is thus not excluded that more than one of a linear alkyl residue, a branched alkyl residue, a cyclic alkyl residue and an aromatic residue, all of which can comprise one or more heteroatoms selected from O, N, S and P, are combined, e.g. an aromatic residue, an alkyl residue, and a heterocyclic residue, or an alkyl residue and a heteroaromatic residue, etc. Particularly, also the alkyl residue can also comprise one or more heteroatoms selected from O, N, S, P, thus also forming e.g. polyethers.

Hydrosilanes are not particularly limited in the context of this invention, a hydrosilane being a compound with at least one Si—H bond. Hydrosilanes are tetravalent silicon compounds containing one or more Si—H bond. Other bonds can comprise organic residues as well as inorganic residues, particularly halogens like F, Cl, Br, I, and/or pseudohalogens.

As used herein, the term “chiral” refers to compounds which have at least one stereocenter. Before the invention is described in exemplary detail, it is to be understood that this invention is not limited to the particular component parts of the process steps of the methods described herein as such methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. For example, the term “a” as used herein can be understood as one single entity or in the meaning of “one or more” entities. It is also to be understood that plural forms include singular and/or plural referents unless the context clearly dictates otherwise. It is moreover to be understood that, in case parameter ranges are given which are delimited by numeric values, the ranges are deemed to include these limitation values.

In a first aspect, the invention relates to a method of producing nornicotine from myosmine. The method comprises reacting myosmine with at least one hydrosilane in the presence of, respectively using, a catalyst, wherein the catalyst is a catalyst of Formula IIa-10 as an organic chiral Lewis base

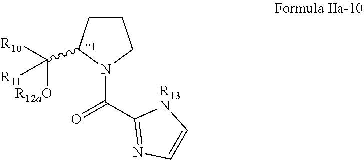

wherein in Formula IIa-10
R₁₀ and R₁₁ are the same or different and are chosen from H; F; Cl; Br; I; pseudohalogen groups; linear alkyl groups with 1 to 10 C atoms that can optionally be substituted with at least one of F, Cl, Br, I, pseudohalogen groups, alkoxy groups with 1 to 20 C atoms, OH, NO₂ and NH₂; branched alkyl groups with 3 to 10 C atoms that can optionally be substituted with at least one of F, Cl, Br, I, pseudohalogen groups, alkoxy groups with 1 to 20 C atoms, OH, NO₂ and NH₂; aralkyl groups with 7 to 24 C atoms that can optionally be substituted with at least one of F, Cl, Br, I, pseudohalogen groups, alkoxy groups with 1 to 20 C atoms, OH, NO₂ and NH₂; alkylaryl groups with 7 to 24 C atoms that can optionally be substituted with at least one of F, Cl, Br, I, pseudohalogen groups, alkoxy groups with 1 to 20 C atoms, OH, NO₂ and NH₂; cycloalkyl groups with 3 to 20 C atoms that can optionally be substituted with at least one of F, Cl, Br, I, pseudohalogen groups, alkoxy groups with 1 to 20 C atoms, OH, NO₂ and NH₂; heterocycloalkyl groups with 1 to 15 C atoms that can optionally be substituted with at least one of F, Cl, Br, I, pseudohalogen groups, alkoxy groups with 1 to 20 C atoms, OH, NO₂ and NH₂; aromatic and/or heteroaromatic groups with 2 to 40 C atoms that can optionally be substituted with at least one of F, Cl, Br, I, pseudohalogen groups, alkoxy groups with 1 to 20 C atoms, OH, NO₂ and NH₂; alkyl heteroaromatic groups with 3 to 50 C atoms that can optionally be substituted with at least one of F, Cl, Br, I, pseudohalogen groups, alkoxy groups with 1 to 20 C atoms, OH, NO₂ and NH₂; heteroaromatic groups with at least one alkyl residue with 3 to 50 C atoms that can optionally be substituted with at least one of F, Cl, Br, I, pseudohalogen groups, alkoxy groups with 1 to 20 C atoms, OH, NO₂ and NH₂; alkyl cycloalkyl groups with 4 to 24 C atoms that can optionally be substituted with at least one of F, Cl, Br, I, pseudohalogen groups, alkoxy groups with 1 to 20 C atoms, OH, NO₂ and NH₂; cycloalkyl alkyl groups with 4 to 24 C atoms that can optionally be substituted with at least one of F, Cl, Br, I, pseudohalogen groups, alkoxy groups with 1 to 20 C atoms, OH, NO₂ and NH₂; alkyl heterocycloalkyl groups with 2 to 24 C atoms that can optionally be substituted with at least one of F, Cl, Br, I, pseudohalogen groups, alkoxy groups with 1 to 20 C atoms, OH, NO₂ and NH₂; heterocycloalkyl alkyl groups with 2 to 24 C atoms that can optionally be substituted with at least one of F, Cl, Br, I, pseudohalogen groups, alkoxy groups with 1 to 20 C atoms, OH, NO₂ and NH₂; carboxylic acid ester groups with 1 to 20 C atoms that can optionally be substituted with at least one of F, Cl, Br, I, pseudohalogen groups, alkoxy groups with 1 to 20 C atoms, OH, NO₂ and NH₂; alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, NO₂ and NH₂; and carboxamide groups with 1 to 20 C atoms that can optionally be substituted with at least one of F, Cl, Br, I, pseudohalogen groups, alkoxy groups with 1 to 20 C atoms, OH, NO₂ and NH₂;
R₁₂ₐ is chosen from H,
linear alkyl groups with 1 to 10 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups (including (CO)NH₂) with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂; linear alkenyl and/or alkynyl groups with 2 to 10 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
branched alkyl, alkenyl and/or alkynyl groups with 3 to 10 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂; aralkyl groups with 7 to 24 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
alkylaryl groups with 7 to 24 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
cycloalkyl groups with 3 to 20 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
heterocycloalkyl groups with 1 to 15 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
aromatic and/or heteroaromatic groups with 2 to 40 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
alkyl heteroaromatic groups with 2 to 50 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
heteroaromatic groups with at least one alkyl residue with 3 to 50 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂; alkyl cycloalkyl groups with 4 to 24 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
cycloalkyl alkyl groups with 4 to 24 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
alkyl heterocycloalkyl groups with 2 to 24 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
heterocycloalkyl alkyl groups with 2 to 24 C atoms that can optionally be substituted with at least one of F, Cl, Br, I, pseudohalogen groups, carboxylic acid ester groups with 1 to 20 C atoms that can optionally be substituted with at least one of F, Cl, Br, I, pseudohalogen groups, alkoxy groups with 1 to 20 C atoms, OH, SH, NO₂ and NH₂, alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, SH, NO₂ and NH₂, carboxamide groups with 1 to 20 C atoms that can optionally be substituted with at least one of F, Cl, Br, I, pseudohalogen groups, alkoxy groups with 1 to 20 C atoms, OH, SH, NO₂ and NH₂, alkoxy groups with 1 to 20 C atoms, OH, SH, NO₂ and NH₂;
SiR₂₃R₂₄R₂₅;
R₂₃, R₂₄, and R₂₅ are the same or different and are selected from H, linear alkyl groups with 1 to 4 C atoms that can optionally be substituted with at least one of F, Cl, Br, I, pseudohalogen groups, alkoxy groups with 1 to 20 C atoms, OH, NO₂ and NH₂, branched alkyl group with 3 to 6 C atoms that can optionally be substituted with at least one of F, Cl, Br, I, pseudohalogen groups, alkoxy groups with 1 to 20 C atoms, OH, NO₂ and NH₂, aromatic groups with 6 to 12 C atoms that can optionally be substituted with at least one of F, Cl, Br, I, pseudohalogen groups, alkoxy groups with 1 to 20 C atoms, OH, NO₂ and NH₂, and aralkyl groups with 7 to 10 C atoms that can optionally be substituted with at least one of F, Cl, Br, I, pseudohalogen groups, alkoxy groups with 1 to 20 C atoms, OH, NO₂ and NH₂, preferably wherein R₂₃, R₂₄, and R₂₅ are not all hydrogen at the same time and/or wherein R₂₃, R₂₄, and R₂₅ are not all methyl at the same time, further preferably wherein all of R₂₃, R₂₄, and R₂₅ are ethyl; and
R₁₃ is chosen from H, linear alkyl groups with 1 to 10 C atoms that can optionally be substituted with at least one of F, Cl, Br, I, pseudohalogen groups, alkoxy groups with 1 to 20 C atoms, OH, NO₂ and NH₂; branched alkyl groups with 3 to 10 C atoms that can optionally be substituted with at least one of F, Cl, Br, I, pseudohalogen groups, alkoxy groups with 1 to 20 C atoms, OH, NO₂ and NH₂; aralkyl groups with 7 to 24 C atoms that can optionally be substituted with at least one of F, Cl, Br, I, pseudohalogen groups, alkoxy groups with 1 to 20 C atoms, OH, NO₂ and NH₂; alkylaryl groups with 7 to 24 C atoms that can optionally be substituted with at least one of F, Cl, Br, I, pseudohalogen groups, alkoxy groups with 1 to 20 C atoms, OH, NO₂ and NH₂; cycloalkyl groups with 3 to 20 C atoms that can optionally be substituted with at least one of F, Cl, Br, I, pseudohalogen groups, alkoxy groups with 1 to 20 C atoms, OH, NO₂ and NH₂; heterocycloalkyl groups with 1 to 15 C atoms that can optionally be substituted with at least one of F, Cl, Br, I, pseudohalogen groups, alkoxy groups with 1 to 20 C atoms, OH, NO₂ and NH₂; aromatic and/or heteroaromatic groups with 2 to 40 C atoms that can optionally be substituted with at least one of F, Cl, Br, I, pseudohalogen groups, alkoxy groups with 1 to 20 C atoms, OH, NO₂ and NH₂; alkyl heteroaromatic groups with 3 to 50 C atoms that can optionally be substituted with at least one of F, Cl, Br, I, pseudohalogen groups, alkoxy groups with 1 to 20 C atoms, OH, NO₂ and NH₂; heteroaromatic groups with at least one alkyl residue with 3 to 50 C atoms that can optionally be substituted with at least one of F, Cl, Br, I, pseudohalogen groups, alkoxy groups with 1 to 20 C atoms, OH, NO₂ and NH₂; alkyl cycloalkyl groups with 4 to 24 C atoms that can optionally be substituted with at least one of F, Cl, Br, I, pseudohalogen groups, alkoxy groups with 1 to 20 C atoms, OH, NO₂ and NH₂; cycloalkyl alkyl groups with 4 to 24 C atoms that can optionally be substituted with at least one of F, Cl, Br, I, pseudohalogen groups, alkoxy groups with 1 to 20 C atoms, OH, NO₂ and NH₂; alkyl heterocycloalkyl groups with 2 to 24 C atoms that can optionally be substituted with at least one of F, Cl, Br, I, pseudohalogen groups, alkoxy groups with 1 to 20 C atoms, OH, NO₂ and NH₂; heterocycloalkyl alkyl groups with 2 to 24 C atoms that can optionally be substituted with at least one of F, Cl, Br, I, pseudohalogen groups, alkoxy groups with 1 to 20 C atoms, OH, NO₂ and NH₂; carboxylic acid ester groups with 1 to 20 C atoms that can optionally be substituted with at least one of F, Cl, Br, I, pseudohalogen groups, alkoxy groups with 1 to 20 C atoms, OH, NO₂ and NH₂; alkyl and/or (hetero)aryl oxycarbonyl groups with 1 to 20 C atoms that can optionally be substituted with at least one of F, Cl, Br, I, pseudohalogen groups, alkoxy groups with 1 to 20 C atoms, OH, NO₂ and NH₂; and carboxamide groups with 1 to 20 C atoms that can optionally be substituted with at least one of F, Cl, Br, I, pseudohalogen groups, alkoxy groups with 1 to 20 C atoms, OH, NO₂ and NH₂;
wherein the stereocenter *1 of the catalyst according to Formula IIa-10 is in (R) and/or (S) configuration. The inventors found that it is surprisingly possible to react myosmine to nornicotine with high efficiency with an organic chiral Lewis base, not requiring metal-containing catalysts.

The reaction conditions are not particularly limited. The conversion of mysomine to nornicotine generally can be described with the following reaction scheme:

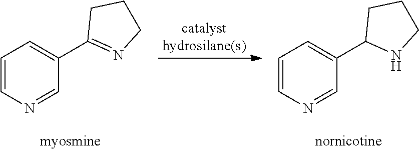 [SMILES: c1cncc(C2=NCCC2)c1 ; c1cncc(C2CCCN2)c1]

The stereocenter *1 of the catalyst according to Formula IIa-10, as well as any of the following formulas directly or indirectly derived therefrom, is in (R) and/or (S) configuration. According to certain embodiments, when the stereocenter *1 of the catalyst of Formula IIa-10 or any of the following formulas in the following is in (R) configuration, (S)-nornicotine is produced in excess. Furthermore, according to certain embodiments, when the stereocenter *1 of the catalyst is in (S) configuration, (R)-nornicotine is produced in excess. According to certain embodiments, when the stereocenter *1 of the catalyst of Formula IIa-10 or any of the following formulas in the following is in (R) configuration, (R)-nornicotine is produced in excess. Furthermore, according to certain embodiments, when the stereocenter *1 of the catalyst is in (S) configuration, (S)-nornicotine is produced in excess.

With the catalytic system described above, the inventors have surprisingly found that it is possible to reduce myosmine, which is a cyclic imine, to nornicotine. It is assumed that the bridge (C═O) of the catalyst according to Formula IIa-10 together with the N in the ring comprising conjugated double bonds are responsible for the coordination of the silane and chemical activation. This allows the hydride transfer from the silane to myosmine, which can be coordinated by the other ring. The stereocenter in ortho position with the residue CR₁₀R₁₁OR₁₂ₐ is thought to direct the coordination of myosmine, which enables its enantioselective hydrosilylation.

According to certain embodiments, R₁₃ is H, a linear alkyl group with 1 to 10, e.g. 1 to 6, C atoms, e.g. a methyl or ethyl group, an aromatic group with 6 to 20 C atoms, e.g. 6 to 10 or 6 C atoms, e.g. a phenyl group, or an aralkyl group with 7 to 20 C atoms, e.g. 7 to 11 C atoms, e.g. 7 C atoms, e.g. a benzyl group. According certain embodiments, the catalyst of Formula IIa-10, is selected from the following Catalysts A1 and A2, and is further preferably Catalyst A1:

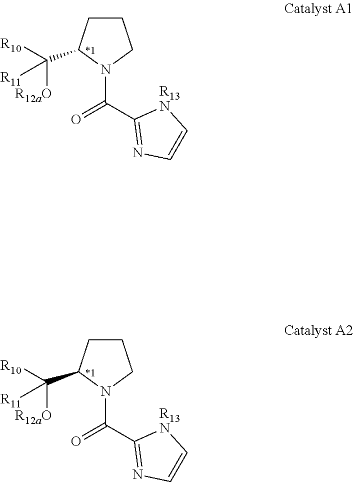

In Catalysts A1 and A2, R₁₀, R₁₁, R₁₂ₐ, and R₁₃ can have the meaning as above.

In Catalysts A1 and A2, R₁₃ is particularly selected from H, alkyl residues with 1 to 4 C atoms, aryl residues with 6 to 12 C atoms, and aralkyl residues with 7 to 14 C atoms, preferably selected from H, methyl, ethyl, phenyl and benzyl;
R₁₀ and R₁₁ are the same or different, but preferably are the same, and are particularly selected from optionally substituted aryl residues with 6 to 12 C atoms, optionally substituted aralkyl residues with 7 to 14 C atoms, and optionally substituted alkylaryl residues with 7 to 14 C atoms that can comprise one or more alkyl group attached to the arlyene unit, wherein the optional substituents can be chosen from halogens like F, Cl, Br, I, and pseudohalogen groups, preferably wherein R₁₀ and R₁₁ are the same and are selected from optionally substituted phenyl, tolyl, naphthyl or xylenyl (e.g. 3,5-xylenyl) groups; and
R₁₂ₐ is selected from H, alkyl groups with 1 to 4 C atoms, alkenyl groups with 1 to 4 C atoms, alkynyl groups with 1 to 4 C atoms, optionally substituted aryl residues with 6 to 12 C atoms, and optionally substituted aralkyl residues with 7 to 14 C atoms, wherein the optional substituents can be chosen from halogens like F, Cl, Br, I, and pseudohalogen groups, preferably methyl, ethyl, 2-propynyl (=propargyl), benzyl and pentafluorobenzyl.

According certain embodiments, the catalyst of Formula IIa-10, is selected from the following Catalysts A-1 to A-LXVI:

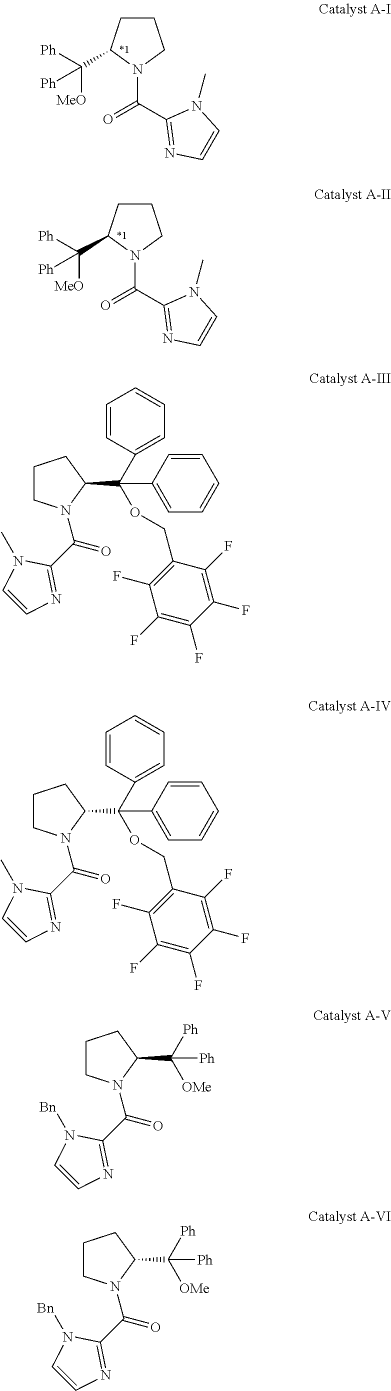

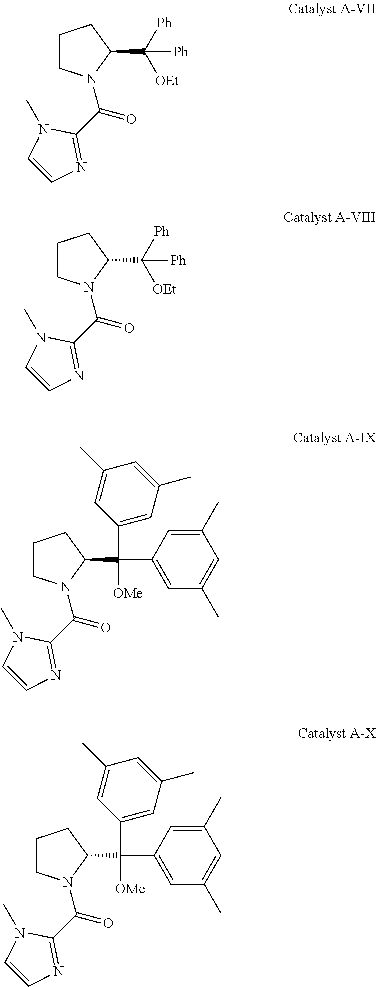

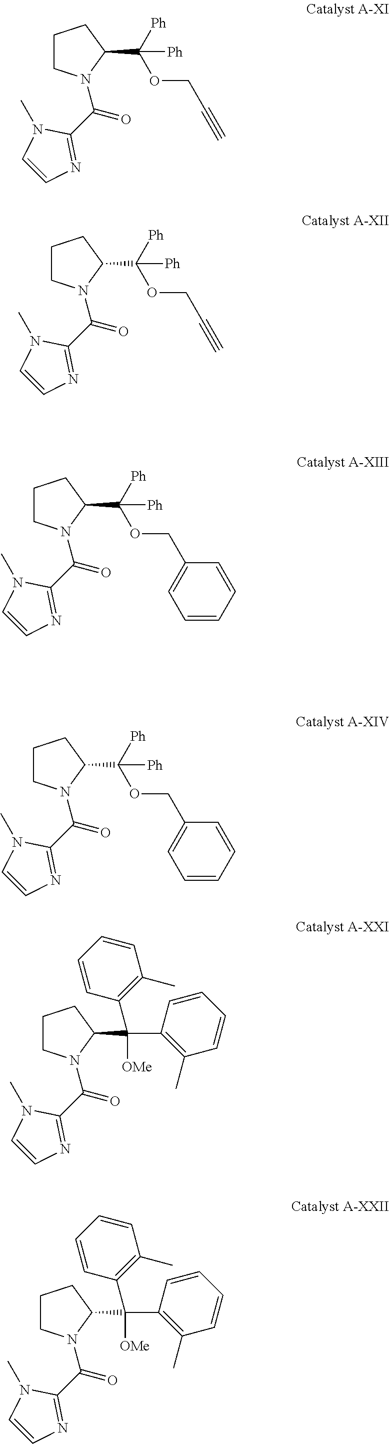

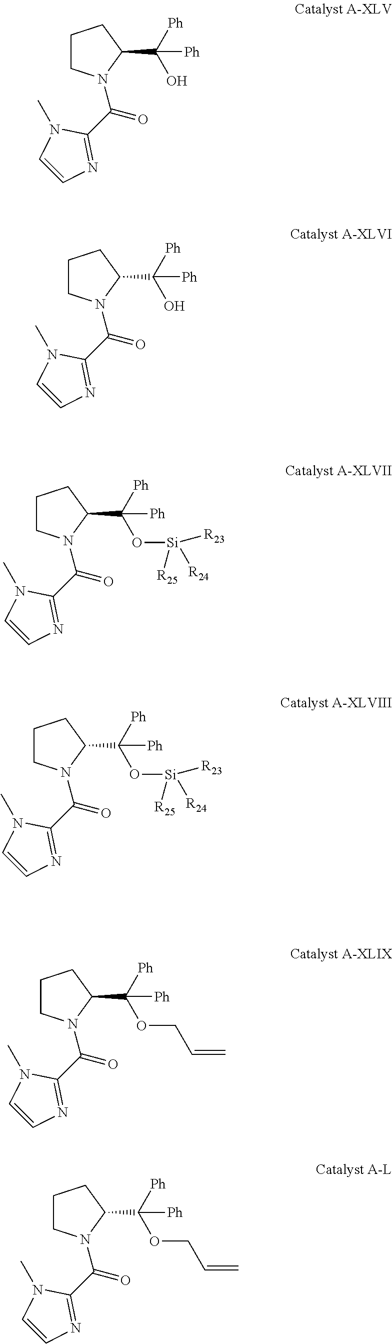

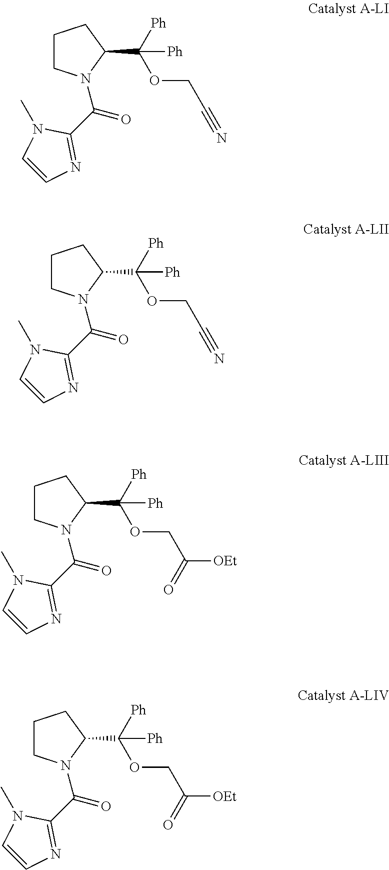 [SMILES: Cn1ccnc1C(=O)N1CCC[C@H]1C(OCC#N)(c1ccccc1)c1ccccc1 ; Cn1ccnc1C(=O)N1CCC[C@@H]1C(OCC#N)(c1ccccc1)c1ccccc1 ; CCOC(=O)COC(c1ccccc1)(c1ccccc1)[C@@H]1CCCN1C(=O)c1nccn1C ; CCOC(=O)COC(c1ccccc1)(c1ccccc1)[C@H]1CCCN1C(=O)c1nccn1C]

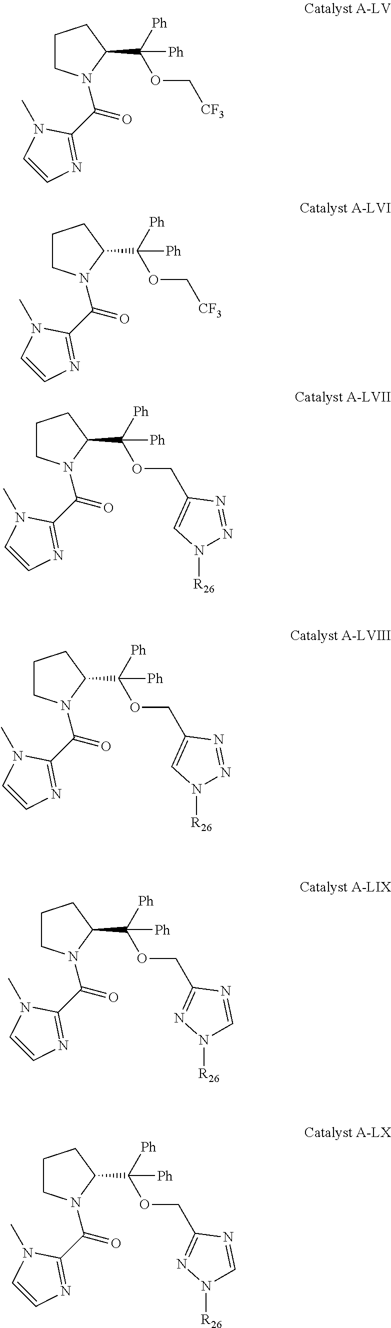

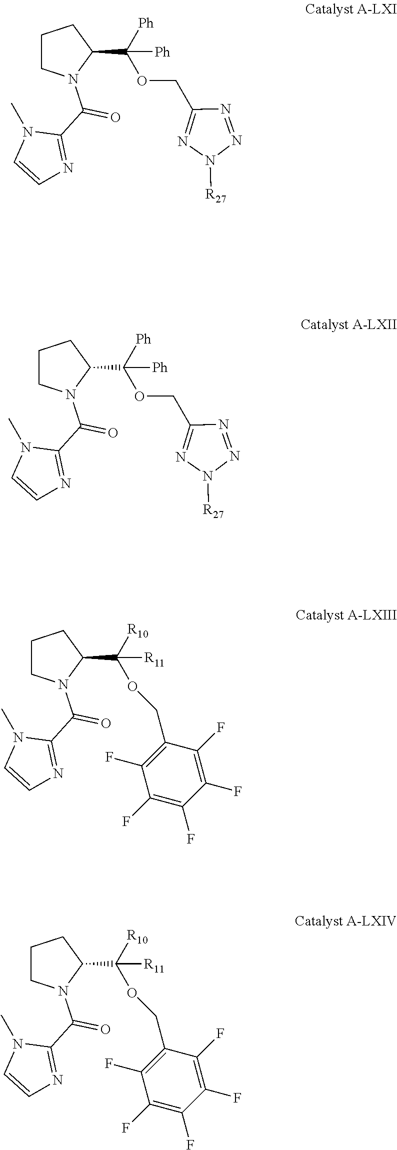

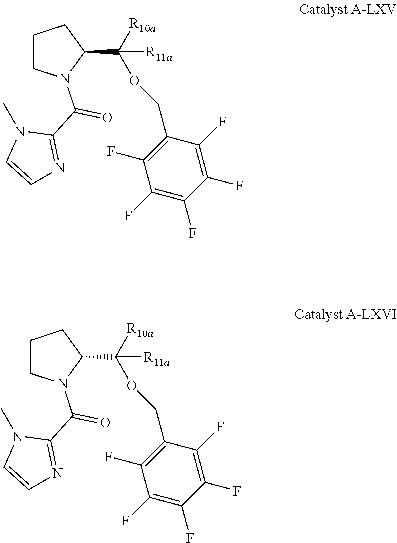

In Catalyst A-XLVII and Catalyst A-XLVIII, R₂₃, R₂₄, and R₂₅ are the same or different and as defined above. According to certain embodiments, R₂₃ and R₂₄ are the same and R₂₅ is different. According to certain embodiments, R₂₃, R₂₄, and R₂₅ are selected from H, linear alkyl groups with 1 to 4 C atoms that can optionally be substituted with at least one of F, Cl, Br, I, pseudohalogen groups, alkoxy groups with 1 to 20 C atoms, OH, NO₂ and NH₂, and branched alkyl group with 3 to 6 C atoms that can optionally be substituted with at least one of F, Cl, Br, I, pseudohalogen groups, alkoxy groups with 1 to 20 C atoms, OH, NO₂ and NH₂. According to certain embodiments, R₂₃ and R₂₄ are Me, Et, or tBu (tert-butyl), e.g. Me, and R₂₅ is Me, Et, or tBu, e.g. tBu. According to certain embodiments, R₂₃ and R₂₄ are Me and R₂₅ is tBu.

In Catalyst A-LVII, Catalyst A-LVIII, Catalyst A-LIX and Catalyst LX, R₂₆ is particularly chosen from H, alkyl residues with 1 to 4 C atoms, aryl residues with 6 to 12 C atoms, and aralkyl residues with 7 to 14 C atoms.

In Catalyst A-LXI and Catalyst A-LXII, R₂₇ is particularly chosen from H, alkyl residues with 1 to 4 C atoms, aryl residues with 6 to 12 C atoms, and aralkyl residues with 7 to 14 C atoms.

In Catalyst A-LXIII and Catalyst A-LXIV, R₁₀ and R₁₁ are as defined above.

In Catalyst A-LXV and Catalyst A-LXVI, R₁₀ₐ and R₁₁ₐ are the same or different, preferably the same, and are selected from the group consisting of optionally substituted aryl residues with 6 to 12 C atoms, optionally substituted aralkyl residues with 7 to 14 C atoms, and optionally substituted alkylaryl residues with 7 to 14 C atoms that can comprise one or more alkyl group attached to the arlyene unit, wherein the optional substituents can be chosen from halogens like F, Cl, Br, I, and pseudohalogen groups, preferably wherein R₁₀ₐ and R₁₁ₐ are the same and are selected from optionally substituted phenyl, tolyl, naphthyl or xylenyl groups.

According to certain embodiments, the catalyst is selected from the group consisting of Catalyst A-I, Catalyst A-II, Catalyst A-Ill, Catalyst A-IV, Catalyst A-V, Catalyst A-VI, Catalyst A-VII, Catalyst A-VIII, Catalyst A-IX, Catalyst A-X, Catalyst A-XI, Catalyst A-XII, Catalyst A-XIII, and Catalyst A-XIV. According to certain embodiments, the catalyst is selected from the group consisting of Catalyst A-I, Catalyst A-Ill, Catalyst A-V, Catalyst A-VII, Catalyst A-IX, Catalyst A-XI, and Catalyst A-XIII. According to certain embodiments, the catalyst is selected from the group consisting of Catalyst A-II, Catalyst A-IV, Catalyst A-VI, Catalyst A-VIII, Catalyst A-X, Catalyst A-XII, and Catalyst A-XIV. According to certain embodiments, the catalyst is selected from the group consisting of Catalyst A-I, Catalyst A-Ill, Catalyst A-V, Catalyst A-VII, Catalyst A-IX, Catalyst A-XI, Catalyst A-XIII, Catalyst A-XXI, Catalyst A-XLV, Catalyst A-XLVII, Catalyst A-XLIX, Catalyst A-LI, Catalyst A-LIII, Catalyst A-LV, Catalyst A-LVII, Catalyst A-LIX, and Catalyst A-LXI. According to certain embodiments, the catalyst is Catalyst A-LXIII.

According to certain embodiments, the catalyst is selected from the group consisting of Catalyst A-II, Catalyst A-IV, Catalyst A-VI, Catalyst A-VIII, Catalyst A-X, Catalyst A-XII, Catalyst A-XIV, Catalyst A-XXII, Catalyst A-XLVI, Catalyst A-XLVIII, Catalyst A-L, Catalyst A-LII, Catalyst A-LIV, Catalyst A-LVI, Catalyst A-LVIII, Catalyst A-LX, and Catalyst A-LXII.

According to certain embodiments, the catalyst is Catalyst A-LXIV.

The temperature of the reaction is not particularly limited. According to certain embodiments, the reaction can be conducted at a temperature in the range of −80° C. to 100° C. In preferred embodiments, the reaction is conducted at a temperature in the range of −30 to 50° C., in particular −20° C. to 25° C. Higher temperatures T promote the competing non-catalyzed hydration reaction, which can lead to low enantiomeric excess ee. Low temperatures can result in very slow reactions with unsatisfactory yields.

The solvent used for the reaction is not particularly limited. The solvent may comprise at least one non-protic solvent or consist essentially of at least one non-protic solvent. In some embodiments, the reaction can be conducted in at least one solvent, selected from the group consisting of dichloromethane (DCM), (trichloromethyl)benzene, (trifluoromethyl)benzene, chloroform, o-xylene, m-xylene, p-xylene, toluene, benzene, chlorobenzene, ethers like anisole and Me-THF, MeCN, acetonitrile, esters like ethyl acetate (EtOAc), acetone and other ketones, e.g. methyl ethyl ketone, methyl isobutyl ketone, methyl isopropyl ketone, cyclopentanone, 2-hexanone, 2- or 3-pentanone, etc., and mixtures of two or more thereof.

According to certain embodiments, the solvent is a fluorinated and/or chlorinated solvent and/or aromatic solvents. According to preferred embodiments, the reaction is conducted in DCM as solvent.

The hydrosilane applied in the reaction is not particularly limited. In certain embodiments, the hydrosilane can have a Si—H bonding strength in the range of 84 to 100 kcal/mol. According to certain embodiments, the hydrosilane can be selected from the group of silanes of formulas ABCSiH, ABSiH₂, and ASiH₃, wherein A, B and C can be the same or different. In certain embodiments, A, B and/or C can be F, Cl, Br and/or I. Alternatively or in addition, A, B and/or C can be alkyl groups with 1 to 20 C atoms that can be substituted with at least one of or two or more of F, Cl, Br, I, pseudohalogen groups, and alkoxy groups with 1 to 20 C atoms.

Alternatively or in addition, A, B and/or C can in some embodiments be aryl groups with 6 to 20 C atoms that can be substituted with at least one of or two or more of F, Cl, Br, I, pseudohalogen groups, and alkoxy groups with 1 to 20 C atoms. In some embodiments, A, B and/or C can, alternatively or in addition, be aralkyl groups with 7 to 24 C atoms that can be substituted with at least one of or two or more of F, Cl, Br, I, pseudohalogen groups, and alkoxy groups with 1 to 20 C atoms. According to certain embodiments, A, B and/or C can, alternatively or in addition, be alkylaryl groups with 7 to 24 C atoms that can be substituted with at least one of or two or more of F, Cl, Br, I, pseudohalogen groups, and alkoxy groups with 1 to 20 C atoms. In preferred embodiments, the hydrosilane is chosen from trichlorosilane (Cl₃SiH), polymethylhydrosiloxane (PHMS), dichlorosilane, and/or methyldichlorosilane, particularly trichlorosilane and/or dichlorosilane, e.g. is particularly trichlorosilane.

According to certain embodiments, the reaction is carried out in the presence of an acid and/or water and/or another protic solvent, which are all not particularly restricted. Exemplary acids include inorganic acids like HCl and organic acids like acetic acid, tartaric acid, malonic acid, p-toluenesulfonic acid, benzoic acid, trifluoroacetic acid. Examples of other protic solvents include alcohols with e.g. 1 to 20 C atoms, e.g. methanol, ethanol, propanol, 1-butanol, 2-butanol, 2,6-Di-tert-butyl-4-methylphenol, etc. It is assumed that these additives help activate the catalyst and/or myosmine, and they may be involved in the regeneration of the catalyst.

The amount of such acid and/or water and/or other protic solvent is not particularly restricted.

The amount of catalyst is not particularly limited. According to certain embodiments, the catalyst can be present in an amount in the range of 0.001 to 60 mol %, preferably 0.005 to 55 mol %, e.g. 0.1 to 50 mol % with respect to the amount of myosmine. In exemplary embodiments, the catalyst is present in an amount in the range of 1 to 20 mol %, e.g. in an amount in the range of 3 to 16 mol %, e.g. in an amount in the range of 7 to 13 mol %, e.g. in an amount of about 10 mol % with respect to the amount of myosmine. For the catalysts, it has been surprisingly found that much lower catalyst amounts are already sufficient. Due to the competitive non-catalyzed reaction, low amounts of catalyst, i.e. lower catalyst concentration c_{c}, lead to lower ee's. It is assumed that the reaction rate of the catalyzed reaction decreases due to the low concentrated catalyst, while the rate of the uncatalyzed reduction remains the same.

The amount of hydrosilane is not particularly limited. In some embodiments, the hydrosilane can be present in an amount in the range of 1 to 10 equivalents with respect to the amount of myosmine. According to some embodiments, the hydrosilane can be present in an amount in the range of 1.2 to 8 equivalents with respect to the amount of myosmine. Further, the hydrosilane can in certain embodiments be present in an amount in the range of 1.5 to 6 equivalents with respect to the amount of myosmine. According to preferred embodiments, hydrosilane is present in an amount in the range of 2 to 5 equivalents, particularly in an amount of about 3.5 equivalents with respect to the amount of myosmine.

The conduction of the reaction is not particularly limited. According to certain embodiments, the nornicotine is produced as a mixture of (R)- and (S)-nornicotine. However, it may also be possible to produce the nornicotine as essentially pure (R)- or (S)-nornicotine with an ee (enantiomeric excess) of at least 99%, e.g. an ee of at least 99.5%, or even as pure (R)- or (S)-nornicotine. According to some embodiments, the reaction can be conducted in liquid phase, e.g. in a solvent as described above. Further, the reaction can be conducted continuously or discontinuously (batch). The reaction may also be conducted in an inert atmosphere which is not particularly restricted.

According to certain embodiments, the nornicotine is produced as a mixture of (R)- and (S)-nornicotine. According to some embodiments, one of the stereoisomers of nornicotine is produced with enantiomeric excess. According to certain embodiments, the enantiomeric excess (ee) of one stereoisomer of nornicotine can be in the range of 5 to less than 100%, in the range of 10 to 99%, or in the range of 20 to 95%. Furthermore, the enantiomeric excess (ee) of one stereoisomer of nornicotine can in certain embodiments be in the range of 50 to 90%. According to certain embodiments, the enantiomeric excess (ee) of (R)-nornicotine can be in the range of 5 to 100%, in the range of 10 to 99%, or in the range of 20 to 95%.

Furthermore, the enantiomeric excess (ee) of (R)-nornicotine can in certain embodiments be in the range of 50 to 90%. According to certain embodiments, the enantiomeric excess (ee) of (S)-nornicotine can be in the range of 5 to 100%, in the range of 10 to 99%, or in the range of to 95%. Furthermore, the enantiomeric excess (ee) of (S)-nornicotine can in certain embodiments be in the range of 50 to 90%.

According to certain embodiments, the method described above can comprise further process steps.

According to certain embodiments, the reaction can be quenched. Quenching is not particularly restricted and allows to break up any complex of the catalyst and product, etc., formed during the reaction to obtain nornicotine, and can be done using a suitable quenching agent like water, a base, an alcohol, etc.

According to certain embodiments, alternatively or in addition to quenching, the obtained reaction mixture can be extracted and/or distilled. Also the extraction and/or distillation is not particularly restricted.

In some embodiments, the method described above further comprises removing the catalyst after reacting myosmine with at least one hydrosilane in the presence of the catalyst. The separation of the catalyst is not particularly limited. The catalyst can easily be separated through various separation methods known in the art. According to some embodiments, the catalyst can be removed by extraction of the reaction mixture, by chromatography, or by filtration upon immobilization, particularly by extraction of the reaction mixture. The extraction is not particularly limited. In certain embodiments, the reaction mixture is extracted with water or an aqueous solution at a specific pH, e.g. pH 7, separating the catalyst into the organic phase and nornicotine into the aqueous phase. According to certain embodiments, the catalyst can be recovered, e.g. through crystallization by concentrating the organic phase, supplementing the organic phase with a suitable solvent like heptane, and cooling the mixture, e.g. to 0° C. or below. It is further not excluded to remove unreacted educt, side products, solvent, and/or removing one enantiomeric nornicotine, e.g. the (S)-nornicotine or the (R)-nornicotine, in order to produce enantiomerically pure nornicotine, e.g. the (R)-nornicotine or the (S)-nornicotine. Techniques for such removal are not particularly restricted and are known in the art.

The method of the first aspect can be carried out continuously or discontinuously, e.g. in a flow reactor, e.g. a microfluidic reactor, or a batch reactor.

In a further, second aspect, the invention relates to a composition produced by the method described above, particularly wherein the catalyst is not removed from the reaction mixture.

Surprisingly it has been found that the reaction mixture with the catalyst can be kept stable, thus allowing also transfer for work-up in bigger batches, saving solvent and energy in the work-up step, e.g. a quench, distillation and/or extraction. Surprisingly, it has been found that the composition produced can be kept stably overnight, nevertheless allowing normal workup afterwards.

In a third aspect, the invention relates to a composition comprising nornicotine and a catalyst, wherein the catalyst is the catalyst as described with regard to the method of the first aspect, and all embodiments described with regard to the catalyst in the first aspect also apply to the third aspect.

According to certain embodiments, the nornicotine can be a mixture of (R)- and (S)-nornicotine, as defined in the first aspect, wherein the enantiomeric excess can be as defined in the first aspect.

In a further, fourth aspect, the invention relates to a method of producing nicotine or a salt thereof from myosmine, comprising reacting myosmine with at least one hydrosilane in the presence of a catalyst to produce nornicotine, wherein the catalyst is the catalyst as described with regard to the method of the first aspect, and all embodiments described with regard to the catalyst in the first aspect also apply to the fourth aspect. Also the first step of the method of the fourth aspect corresponds, according to certain embodiments, to the reaction step of the method of the first aspect. Thus, also the different embodiments described with regard to the method of the first aspect also apply, according to certain embodiments, to the method of the fourth aspect, particularly to the first reaction step, and vice versa.

In a second step, which according to certain embodiments is a subsequent step, the nornicotine is then reacted to nicotine. In a further, according to certain embodiments subsequent step, the nicotine can then be reacted to a salt thereof. The inventors particularly found that the two steps of reacting myosmine to nornicotine and then reacting nornicotine to nicotine can be carried out in one pot, i.e. without any cleaning steps in between. According to some embodiments, the method of the fourth aspect can be conducted according to the following reaction scheme:

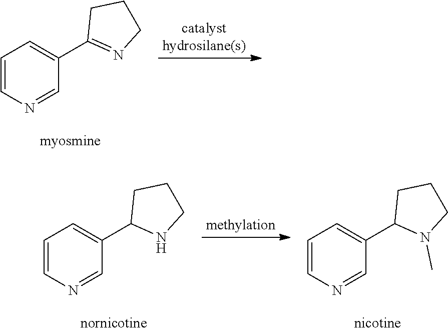

The reaction of myosmine with at least one hydrosilane to produce nornicotine is not particularly limited apart from the presence of an organic chiral Lewis base as catalyst.

According to certain embodiments, the reaction of myosmine with at least one hydrosilane to produce nornicotine corresponds to the respective embodiments in the method of producing nornicotine from myosmine of the first aspect, as described above.

The reaction of nornicotine to nicotine is not particularly limited. In some embodiments, the pyrrolidine ring is methylated at the N-atom by a suitable methylating agent, which also is not particularly restricted. According to certain embodiments, the reaction of nornicotine to nicotine is carried out using formic acid and paraformaldehyde, or formic acid and formaldehyde, preferably at a temperature between and including 40 to 95° C., further preferably between and including 60 to 85° C.

According to certain embodiments, the nicotine is further reacted to a salt thereof. The reaction is not particularly restricted, and any suitable salt can be produced, e.g. a tartrate salt or a salicylate salt or a co-crystal compound thereof with e.g. beta-cyclodextrine.

According to certain embodiments, the method can be carried out in a one-pot process. This saves of course further separating steps, solvents, energy and time. It has been surprisingly found that the whole synthesis can be made as a one-pot process without any purification steps of the intermediates in such embodiments. One specific further advantage of the invention is the one-pot synthesis allowing a straight forward reaction sequence, using minimal amounts and types of solvents. According to certain embodiments, no solvent change is necessary in such a one-pot process. It is, however, not excluded that a further solvent is added, e.g. for removing the catalyst and/or for a subsequent step. According to certain embodiments, the catalyst is separated from the mixture after producing nornicotine by extraction, e.g. with an aqueous solvent, e.g. water, and the remaining reaction mixture is directly converted to yield nicotine, without further work-up of the reaction mixture, thus enabling a one-pot synthesis.

Furthermore, since one enantiomer of nornicotine can be produced enantioselectively through the method described above, the corresponding enantiomer of nicotine can also be synthesized with high enantiomeric excess without the application of a resolution step.

According to certain embodiments, the nicotine is produced as a mixture of (R)- and (S)-nornicotine, as defined in the first aspect, wherein the enantiomeric excess can be as defined in the first aspect.

In a further, fifth aspect, the invention relates to a composition produced by the method of producing nicotine or a salt thereof from myosmine as described above, wherein the catalyst is not separated from the reaction mixture. As the composition of the fifth aspect directly relates to the fourth aspect, also the corresponding embodiments of the first, and also second, aspect apply. Herein, again, it has been surprisingly found that the mixture can be kept stable, so that a work-up of several batches is possible, thus saving e.g. solvent(s) and energy. Surprisingly, it has been found that the composition produced can be kept stably overnight, nevertheless allowing normal workup afterwards.

In a further, sixth aspect, the invention relates to a composition comprising nicotine or a salt thereof and a catalyst, wherein the catalyst is the catalyst as described with regard to the method of the first aspect, and all embodiments described with regard to the catalyst in the first aspect also apply to the sixth aspect.

According to certain embodiments, the nicotine or the salt thereof can a be mixture of (R)- and (S)-nicotine or the salts thereof, as defined in the first aspect, wherein the enantiomeric excess can be as defined in the first aspect.

The above embodiments can be combined arbitrarily, if appropriate. Further possible embodiments and implementations of the invention comprise also combinations of features not explicitly mentioned in the foregoing or in the following with regard to the Examples of the invention. Particularly, a person skilled in the art will also add individual aspects as improvements or additions to the respective basic form of the invention.

### EXAMPLES

The present invention will now be described in detail with reference to several examples thereof. However, these examples are illustrative and do not limit the scope of the invention.

### Reference Examples A: Catalyst Synthesis

### 1) Catalyst A-1

Catalyst A-I was obtained commercially (Fluorochem Ltd.).

Catalyst A-1 was alternatively obtained from A-XLV (described below). A-XLV was dissolved in dry DMF (7 mL) under N₂ atmosphere and precooled to −10° C. NaH (60% dispersion in mineral oil, 49.8 mg, 1.25 mmol) was added at −10° C. and stirred for 0.5 h at rt. Mel (77.5 μL, 1.25 mmol) was added dropwise to the reaction mixture and stirred at RT for 4 h. The reaction mixture was quenched using sat. aq. NH₄Cl (10 mL). The aqueous phase was extracted with ethyl acetate (3×15 mL) and the combined organic phases were washed with H₂O (2×15 mL) and dried over Na₂SO₄ and concentrated under reduced pressure. The crude product was purified by column chromatography (gradient: MeCN/H₂O 6:4 in 4 CV to 9:1) to give the catalyst as a colourless solid (0.265 g, 85%). The ¹H- and ¹³C-NMR spectra are matching the spectra published in Org. Biomol. Chem. 2017, 15, 2422-2435.

2) Catalyst A-V (S)-(1-benzyl-1H-imidazol-2-yl)(2-(methoxydiphenylmethyl)pyrrolidin-1-yl)methanone

(S)-diphenyl(pyrrolidin-2-yl)methanol (AB171959|CAS 112068-01-6-abcr Gute Chemie) (1.18 g, 4.66 mmol) was dissolved in dry toluene (20 mL) and NaH (60% dispersion in mineral oil, 0.242 g, 6.06 mmol) was added to it at room temperature (rt). After stirring the mixture for 0.5 h at rt, ethyl 1-benzyl-1H-imidazole-2-carboxylate (prepared according to Eur. J. Med. Chem. 2022, 228, 113965 from commercially available building blocks (Sigma Aldrich)) (1.20 g, 5.55 mmol) was added. The reaction mixture was heated to 70° C. and stirred at the same temperature for another 2.5 h. After cooling the reaction mixture back to rt, the reaction was quenched with saturated aqueous NH₄Cl (30 mL). The phases were separated and the aqueous phase was extracted three times with dichloromethane (3×30 mL). The combined organic layers were washed with water (3×50 mL), dried over Na₂SO₄ and concentrated under reduced pressure. The resulting oily crude product was dissolved in ethyl acetate/heptane 7:3 (3 mL) and filtered over a short silicagel column using more ethyl acetate/heptane (7:3). The filtrate was concentrated under reduced pressure to give 1.60 g (˜79%) of yellowish solid, which was used for the next step without further purification. 0.900 g (2.06 mmol) of the crude product was dissolved in dry DMF (16 mL), the solution was cooled to 0° C. and NaH (60% dispersion in mineral oil, 0.123 g, 3.09 mmol) was added to it. The reaction mixture was stirred for 0.5 h at 0° C. and then let warm to rt. Mel (0.192 mL, 3.09 mmol) was added dropwise and the yellow solution was stirred at rt for another 4 h. The reaction was quenched by the addition of sat. aq. NH₄Cl (15 mL). The aqueous layer was extracted with ethyl acetate (3×25 mL). The combined organic layers were washed with water (2×25 mL), dried over Na₂SO₄ and concentrated under reduced pressure. The crude product was purified by column chromatography (ethyl acetate/heptane 45:55) to give the product (0.806 g, 69% after two steps) as a white solid. Two rotamers were observed in NMR in CDCl₃ with a ratio of 1.00:0.39.

¹H NMR (400 MHz, CDCl₃, rotamer A) δ 7.52-7.28 (m, 14H), 7.21-7.13 (m, 1H), 6.98 (s, 1H), 6.76 (s, 1H), 6.46 (dd, J=7.83 Hz, 1H), 5.65-5.53 (m, 1H), 5.14 (d, J=14.87 Hz, 1H), 3.60-3.59 (m, 1H), 2.50-2.33 (m, 2H), 2.33 (s, 3H), 2.10-1.90 (m, 1H), 1.68-1.58 (m, 1H), 1.11-1.07 (m, 1H). ¹H NMR (400 MHz, CDCl₃, rotamer B) δ 7.52-7.28 (m, 15H), 7.09 (s, 1H), 6.92 (s, 1H), 5.65-5.53 (m, 2H), 5.37-5.30 (m, 1H), 3.50-3.49 (m, 1H), 3.03 (s, 3H), 2.99-2.84 (m, 1H), 2.10-1.90 (m, 2H), 1.43-1.19 (m, 1H), 1.11-1.07 (m, 1H). ¹³C NMR (101 MHz, CDCl₃, rotamer A) δ 161.0, 142.4, 139.9, 138.9, 136.7, 130.2, 129.5 (multiple signals), 128.8 (multiple signals), 127.7 (multiple signals), 127.1, 121.1, 86.9, 63.4, 51.8, 51.0, 47.6, 27.7, 21.9. ¹³C NMR (101 MHz, CDCl₃, rotamer B) δ 161.5, 141.3, 141.1, 140.0, 137.4, 130.0, 129.7 (multiple signals), 128.8 (multiple signals), 127.7 (multiple signals), 127.5, 122.6, 86.7, 60.6, 52.6, 51.1, 50.6, 26.7, 23.8. LC-MS: [M-OMe]+=420.2 m/z, [M+H]⁺=452.2 m/z, calculated mass 451.2 Da.

4) Catalyst A-XLV (S)-(2-(hydroxydiphenylmethyl)pyrrolidin-1-yl)(1-methyl-1H-imidazol-2-yl)methanone

Catalyst A-XLV was prepared according to the procedure described in Org. Biomol. Chem. 2017, 15, 2422-2435 and purified by crystallization from EtOAc/heptane. Catalyst A-XLV was obtained as a colourless solid in 86% yield. LC-MS: [M-OH]⁺=344.2 m/z, [M+H]⁺=362.2 m/z, calculated MW 361.2

5) Catalyst A-VII (S)-(2-(ethoxydiphenylmethyl)pyrrolidin-1-yl)(1-methyl-1H-imidazol-2-yl)methanone

Catalyst A-VII was prepared analogously to catalyst Catalyst A-1. Catalyst A-XLV (1.00 g, 2.77 mmol), NaH (60% dispersion in mineral oil, 0.166 g, 4.15 mmol) and Etl (0.335 mL, 4.15 mmol) gave Catalyst A-VII as a colourless solid (0.320 g, 30%). Two rotamers were observed in NMR in CDCl₃ with a ratio of 1.00:0.21.

¹H NMR (400 MHz, CDCl₃, rotamer A) δ 7.39-7.23 (m, 10H), 6.97 (s, 1H), 6.77 (s, 1H), 6.68 (dd, J=9.00, 1.39 Hz, 1H), 3.74 (s, 3H), 3.64 (ddd, J=12.17, 9.63, 5.32 Hz, 1H), 2.90-2.83 (m, 1H), 2.57-2.43 (m, 2H), 2.36-2.29 (m, H), 2.05-1.96 (m, 1H), 1.67-1.57 (m, 1H), 1.24-1.16 (m, 1H), 0.76 (t, J=6.97 Hz, 3H). ¹H NMR (400 MHz, CDCl₃, rotamer B) δ 7.52 (d, J=7.10 Hz, 2H), 7.46 (d, J=7.10 Hz, 2H), 7.39-7.23 (m, 6H), 7.07 (s, 1H), 6.87 (s, 1H), 5.68 (dd, J=1.00 Hz, 1H), 3.82 (s, 3H), 3.74-3.82 (m, 1H), 3.40-3.36 (m, 1H), 3.18-3.10 (m, 1H), 2.90-2.83 (m, 1H), 2.36-2.29 (m, 1H), 2.05-1.96 (m, 1H), 1.67-1.57 (m, 1H), 1.24-1.16 (m, 1H), 1.10 (t, J=6.84. Hz, 3H). ¹³C NMR (101 MHz, CDCl₃, rotamer A) δ 160.7, 142.1, 139.2, 138.8, 129.7 (multiple signals), 127.7 (multiple signals), 127.6, 126.5, 122.4, 86.7, 65.2, 58.9, 47.3, 34.9, 27.5, 21.9, 14.9. ¹³C NMR (101 MHz, CDCl₃, rotamer B) δ 160.9, 141.7, 140.9 140.7, 129.7 (multiple signals), 127.7 (multiple signals), 127.6 (multiple signals), 123.8, 86.3, 61.2, 59.6, 50.7, 35.4, 26.6, 24.1, 15.5. LC-MS: [M-OEt]⁺=344.2 m/z, [M+H]⁺=390.2 m/z, calculated mass 389.2 Da

6) Catalyst A-IX (S)-(2-(bis(3,5-dimethylphenyl)(methoxy)methyl)pyrrolidin-1-yl)(1-methyl-1H-imidazol-2-yl)methanone

Catalyst A-IX was prepared analogously to catalyst Catalyst A-V. (S)-bis(3,5-dimethylphenyl)(pyrrolidin-2-yl)methanol (Sigma Aldrich) (0.500 g, 1.62 mmol), dry toluene (5 mL), NaH (60% dispersion in mineral oil, 0.0840 g, 2.11 mmol) and ethyl-1-methyl-1H-imidazole-2-carboxylate (AB245872 I CAS 30148-21-1-abcr Gute Chemie) (0.296 g, 1.92 mmol) gave (S)-(2-(bis(3,5-dimethylphenyl)(hydro-xy)methyl)pyrrolidin-1-yl)(1-methyl-1H-imidazol-2-yl)methanone as a white solid (0.675 g, ˜81%) after purification with column chromatography (heptane/EtOAc, gradient 35-100%). 0.544 g (1.30 mmol) of the purified product, NaH (60% dispersion in mineral oil, 0.0782 g, 1.95 mmol), DMF (8 mL) and Mel (0.122 mL, 1.95 mmol) yielded the catalyst (0.354 g, 59%) as a white solid after purification with reverse phase column chromatography (MeCN/H₂O 60-90%). Two rotamers were observed in NMR in CDCl₃ with a ratio of 1.00:0.27.

¹H NMR (400 MHz, CDCl₃, rotamer A) δ 7.11 (br d, J=7.83 Hz, 1H) 7.06-6.86 (m, 6H), 6.80 (s, 1H), 6.18 (d, J=7.83 Hz, 1H), 3.84 (s, 3H), 3.58-3.51 (m, 1H), 2.37-2.31 (m, 2H), 2.33 (s, 3H), 2.30 (s, 6H), 2.26 (s, 6H), 2.01-1.99 (m, 1H), 1.60-1.56 (m, 1H), 1.08-1.06 (m, 1H). ¹H NMR (400 MHz, CDCl₃, rotamer B) δ 7.06-6.86 (m, 8H), 5.60 (br dd, J=9.00, 3.00 Hz 1H), 3.90 (s, 3H), 3.78-3.65 (m, 1H), 2.98 (s, 3H), 2.78-2.88 (m, 1H), 2.43-2.33 (m, 1H), 2.30 (s, 6H), 2.26 (s, 6H), 2.01-1.99 (m, 1H), 1.60-1.56 (m, 1H), 1.08-1.06 (m, 1H). ¹³C NMR (101 MHz, CDCl₃, rotamer A) δ 161.0, 142.9, 140.1, 139.1, 137.1, 136.9, 136.7, 129.4 (multiple signals), 129.1 (multiple signals), 128.0, 127.4, 126.7, 122.4, 86.9, 63.4, 51.7, 47.3, 34.2, 28.0, 21.9, 21.7, 21.6.13C NMR (101 MHz, CDCl₃, rotamer B) δ 161.0, 141.1, 140.9, 140.9, 136.8 (multiple signals), 129.4 (multiple signals), 129.1 (multiple signals), 127.9, 127.4, 127.1, 124.0, 87.3, 60.6, 52.5, 50.9, 35.5, 26.8, 24.2, 21.7, 21.8. LC-MS: [M-OMe]⁺=400.2 m/z, [M+H]⁺=432.3 m/z, calculated mass 431.3 Da.

7) Catalyst A-XLVII (R₂₃, R₂₄=methyl, R₂₅=t-butyl) (S)-(2-((tert-butyldimethylsilyloxy)diphenylmethyl)pyrrolidin-1-yl)(1-methyl-1H-imidazol-2-yl)methanone

Catalyst A-XLV (0.200 g, 0.553 mmol) was dissolved in dry DCM (1.0 mL) under N₂ atmosphere and precooled to 0° C. 2,6-Lutidine (0.446 mL, 3.83 mmol) and tert-butyldimethylsilyl triflate (TBSOTf) (0.625 mL, 2.72 mmol) were added to it at 0° C. and the reaction was let warm up to rt. After stirring the reaction mixture for 5 hours at the same temperature, it was quenched with sat. aq. NH₄Cl (1 mL). The layers were separated and the aqueous layer was extracted with EtOAc (3×3 mL). The combined organic layers were washed with 9% aq. NaHCO₃ (3×3 mL), then with H₂O (2×3 mL) and finally dried over Na₂SO₄ and concentrated under reduced pressure. The crude product was purified by column chromatography (EtOAc/Heptane: 30% EtOAc isocratic) to give the product as a colourless oil (0.222 g, 85%). Two rotamers were observed in NMR in CDCl₃ with a ratio of 1.00:0.87.

¹H NMR (400 MHz, CDCl₃, rotamer A) δ 7.32-7.14 (m, 10H), 6.91 (br s, 1H), 6.69 (br s, 2H), 3.82-3.70 (m, 1H), 3.25 (s, 3H), 3.20-3.13 (m, 1H), 2.31-2.25 (m, 1H), 2.11-2.07 (m, 1H), 1.91-1.77 (m, 1H), 1.67-1.61 (m, 1H), 0.88 (s, 9H), −0.23 (s, 3H), −0.51 (s, 3H). ¹H NMR (400 MHz, CDCl₃, rotamer B) δ 7.64-7.55 (m, 10H), 7.02 (br s, 1H), 6.86 (br s, 1H), 5.81 (d, J=6.87 Hz, 1H), 4.00-3.92 (m, 1H), 3.82 (s, 3H), 3.01-2.93 (m, 1H), 2.58-2.51 (m, 2H), 1.67-1.61 (m, 1H), 1.13-1.10 (m, 1H), 0.93 (s, 9H), −0.34 (s, 3H), −0.40 (s, 3H). ¹³C NMR (400 MHz, CDCl₃, rotamer A) δ 159.7, 144.1, 140.9 (multiple signals), 130.5 (multiple signals), 130.2 (multiple signals), 128.7 (multiple signals), 127.8 (multiple signals), 127.7 (multiple signals), 126.5, 123.2, 86.0, 65.4, 47.6, 34.9, 26.4, 26.4, 22.9, 19.2, −2.23, −3.1 (multiple signals). ¹³C NMR (400 MHz, CDCl₃, rotamer B) δ 161.7, 143.6, 140.9 (multiple signals), 130.5 (multiple signals), 130.2 (multiple signals), 128.7 (multiple signals), 127.8 (multiple signals), 127.7 (multiple signals), 126.5, 124.0, 84.6, 62.4, 51.2, 35.5, 28.2, 26.4, 24.2, 14.3, −2.2, −3.1 (multiple signals). LC-MS: [M-OTBMS]⁺=344.2 m/z, calculated mass 475.3 Da

8) Catalyst A-XI (S)-(2-(diphenyl(prop-2-ynyloxy)methyl)pyrrolidin-1-yl)(1-methyl-1H-imidazol-2-yl)methanone

Catalyst A-XI was synthesized analogously to Catalyst A-1. Catalyst A-XLV (0.500 g, 1.39 mmol) in DMF (9 mL), NaH (60% dispersion in mineral oil, 0.0830 g, 2.08 mmol) and propagyl bromide (80% in toluene, 0.231 mL, 2.08 mmol) gave the product as a yellow oil (0.161 g, 29%) after the purification with reverse phase column chromatography (MeCN/H₂O 60-90%). Two rotamers were observed in NMR in CDCl₃ with a ratio of 1.00:0.21. ¹H NMR (400 MHz, CDCl₃, rotamer A) δ 7.56-7.50 (m, 1H), 7.47-7.29 (m, 9H), 6.99 (s, 1H), 6.80 (s, 1H), 6.61 (d, J=9.06 Hz, 1H), 3.81 (s, 3H), 3.70-3.53 (m, 1H), 3.41 (dd, J=17.32, 2.63 Hz, 1H), 3.12 (d, J=17.15, 2.10 Hz, 1H), 2.53-2.43 (m, 1H), 2.42-2.30 (m, 1H), 2.29-2.19 (m, 1H), 2.12-1.90 (m, 1H), 1.75-1.53 (m, 1H), 1.35-1.12 (m, 1H). ¹H NMR (400 MHz, CDCl₃, rotamer B) δ 7.47-7.29 (m, 10H), 7.08 (s, 1H), 6.91 (s, 1H), 5.68-5.61 (m, 1H), 4.16 (d, J=15.40 Hz, 1H), 3.91-3.87 (m, 1H), 3.87 (s, 3H), 3.57-3.63 (m, 1H), 2.29-3.18 (m, 1H), 2.42-2.30 (m, 1H) 2.29-2.19 (m, 1H), 2.12-1.90 (m, 1H), 1.75-1.53 (m, 1H), 1.35-1.12 (m, 1H). ¹³C NMR (101 MHz, CDCl₃, rotamer A) δ 160.9, 142.0, 138.2, 138.0, 130.0, 129.6 (multiple signals), 128.1 (multiple signals), 127.9 multiple signals), 126.5, 123.0, 87.8, 80.5, 72.8, 64.7, 52.6, 47.4, 35.2, 27.7, 21.9. ¹³C NMR (101 MHz, CDCl₃, rotamer B) b 161.1, 141.0, 139.5 (signals), 130.1 (multiple signals), 129.9 (multiple signals), 128.4, 128.2, 125.4, 124.0, 87.3, 81.3, 72.9, 61.2, 53.2, 50.8, 35.6, 26.8, 24.1. LC-MS: [M-O-propargyl]⁺=344.2 m/z, [M+H]⁺=400.2 m/z, calculated mass 399.2 Da.

9) Catalyst A-XIII (S)-(2-(benzyloxydiphenylmethyl)pyrrolidin-1-yl)(1-methyl-1H-imidazol-2-yl)methanone

Catalyst A-XIII was synthesized analogously to Catalyst A-V. Catalyst A-XLV (0.500 g, 1.39 mmol) in DMF (9 mL), NaH (60% dispersion in mineral oil, 0.0830 g, 2.08 mmol) and benzyl bromide (0.246 mL, 2.08 mmol) gave the product as a yellow oil (0.385 g, 62%) after the purification with reverse phase column chromatography (MeCN/H₂O 60-90%). Two rotamers were observed in NMR in CDCl₃ with a ratio of 1.00:0.16.

¹H NMR (400 MHz, CDCl₃, rotamer A) δ 7.71-7.23 (m, 15H), 6.98 (s, 1H), 6.71 (d, J=9.10 Hz, 1H), 6.51 (s, 1H), 3.84 (d, J=10.85 Hz, 1H), 3.65-3.55 (m, 1H), 3.53 (d, J=11.20 Hz, 1H), 3.19 (s, 3H), 2.48-2.32 (m, 2H), 2.20-1.95 (m, 1H), 1.74-1.55 (m, 1H), 1.27-1.08 (m, 1H). ¹H NMR (400 MHz, CDCl₃, rotamer B) δ 7.71-7.23 (m, 11H), 7.08 (s, 1H), 6.95-6.90 (m, 4H), 6.89 (s, 1H), 5.82 (d, J=7.70 Hz, 1H), 4.53 (d, J=12.25 Hz, 1H), 4.02 (d, J=10.85 Hz, 1H), 3.90-3.87 (m, 1H), 3.78 (s, 3H), 2.20-1.95 (m, 4H), 1.35-1.27 (m, 1H). ¹³C NMR (101 MHz, CDCl₃, rotamer A) δ 160.9, 142.0, 138.8, 138.8, 138.6, 130.0 (multiple signals), 128.1 (multiple signals), 127.9 (multiple signals), 127.8, 127.3, 126.1, 123.2, 87.4, 66.1, 64.9, 47.5, 34.1, 27.6, 21.9. ¹³C NMR (101 MHz, CDCl₃, rotamer B) δ 161.0, 141.4, 140.9, 140.4, 139.7, 130.0 (multiple signals), 128.1 (multiple signals), 127.9 (multiple signals), 127.8, 127.3, 127.1, 124.0, 87.0, 66.1, 61.6, 50.7, 35.5, 26.7, 24.2. LC-MS: [M-O-benzyl]⁺=344.2 m/z, [M+H]⁺=452.3 m/z, calculated mass 451.2 Da.

10) Catalyst A-Ill (S)-(1-methyl-1H-imidazol-2-yl)(2-((perfluorobenzyloxy)diphenylmethyl)-pyrrolidin-1-yl)methanone

Catalyst A-Ill was produced analogously to Catalyst A-V. Catalyst A-XLV (0.800 g, 2.21 mmol, 1.00 equiv.), NaH (60% dispersion in mineral oil, 0.133 g, 3.32 mmol, 1.50 equiv.), 2,3,4,5,6-pentafluorobenzyl bromide solution (0.501 mL, 3.32 mmol, 1.50 equiv.) and DMF (14 mL) gave Catalyst A-Ill as a white solid (0.366 g, 36%). Two rotamers were observed in NMR in CDCl₃ with a ratio of 1.00:0.21.

¹H NMR (400 MHz, CDCl₃, rotamer A) δ 7.66-7.23 (m, 10H), 6.93 (s, 1H), 6.73 (d, J=9.22 Hz, 1H), 6.46 (s, 1H), 4.03 (d, J=9.98 Hz, 1H), 3.67-3.42 (m, 2H), 3.53 (s, 3H), 2.45-2.29 (m, 1H), 2.29-2.15 (m, 1H), 2.01-1.89 (m, 1H), 1.71-1.46 (m, 1H), 1.21-1.05 (m, 1H). ¹H NMR (400 MHz, CDCl₃ rotamer B) δ 7.66-7.23 (m, 10H), 7.06 (s, 1H), 6.89 (s, 1H), 6.73 (m, 1H), 4.70 (d, J=11.01 Hz, 1H), 4.12 (m, 1H), 3.88-3.75 (m, 1H), 3.83 (s, 3H), 3.11-3.00 (m, 1H), 2.45-2.29 (m, 1H), 2.15-2.06 (m, 1H), 1.71-1.46 (m, 1H), 1.21-1.05 (m, 1H). ¹³C NMR (400 MHz, CDCl₃, due to the complexity of the spectrum, it was not possible to assign the specific peaks to rotamer A or B and the peaks are reported altogether) δ 161.1, 160.0-159.3 (multiplet), 147.1-146.7 (multiplet), 144.6-144.0 (multiplet), 142.6-141.9 (multiplet), 141.5-141.0 (multiplet), 140.7, 140.0, 139.2-139.8 (multiplet), 138.4-138.5 (multiplet), 137.6, 136.8, 136.3-135.7 (multiplet), 130.1 (multiple signals), 129.7 (multiple signals), 128.5, 128.2 (multiple signals), 127.8, 127.7, 127.6, 125.2-126.1, 124.2, 122.2, 111.7 (td, J=18.2, 3.6 Hz), 88.4, 87.3, 65.7, 60.8, 54.4, 53.3, 50.8, 47.5, 35.5, 34.8, 27.7, 26.9, 24.0, 21.9.

### 11) Further Catalysts

For further studies, also some further catalysts were synthesized without actually being tested afterwards. Catalyst A-XLIX was prepared analogously to Catalyst A-VI. Catalyst A-LVII was prepared from Catalyst A-XI (described in point 8) by CuAAC (copper (I)-catalyzed alkyne-azide cycloaddition).

### Reference Examples C: Reference Catalysts

For Comparative Examples, Catalyst C and Catalyst D were provided.

Catalyst C was obtained commercially.

Catalyst D was synthesized according to its published procedure (Adv. Synth. Catal. 2008, 350, 619-623) and ¹H NMR is matching the published spectra.

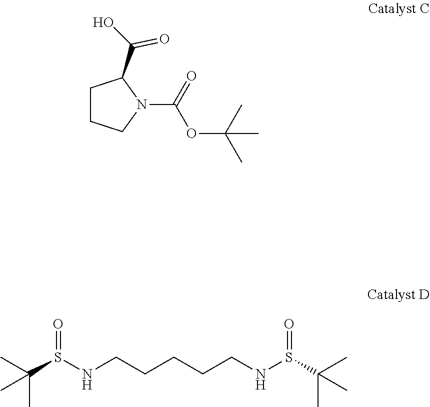

### Example A-1A: Synthesis of (R/S)-Nornicotine

The synthesis is carried out according to the following general reaction:

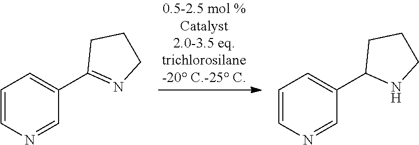 [SMILES: c1cncc(C2=NCCC2)c1 ; c1cncc(C2CCCN2)c1]

1 eq myosmine (100 mg; 305 mg in entry A4-A-9) and the respective catalyst (mol %) with varying amounts, as given in Table 1 below, are dissolved in DCM (dried) as solvent, as given in Table 1. In some experiments, also an additive (benzoic acid) was given, as some additives showed a further improvement in results, as discussed above and also shown below. Different equivalents (eq.) of trichlorosilane, as given in Table 1 below, are added at different reaction temperatures as given in Table 1 below, and the solution is stirred for 18-22 h. After the reaction time, 8-30% NaOH in water is added until all solids solubilize and the mixture is extracted twice with 40 mL DCM each. The organic phases are collected, dried, filtered and reduced in vacuo, resulting in a mixture of (R/S)-nornicotine. The enantiomeric excess is determined by HPLC (mobile phase hexane:isopropanol=95:5 1 mL/min, stationary phase Daicel Chiralcel OD-H 25 cm×0.45 cm, detection wavelength 220 nm). The results of different Examples are given in Table 1.

TABLE 1
Results of a series of examples with varying catalysts, amounts of catalyst,
temperature, silane loading equivalents, and additive, following the procedure described
above. The enantiomeric excess refers to (S)-nornicotine.

| | | Catalyst | Myosmine | | Silane | | | |
|---|---|---|---|---|---|---|---|---|
| | | concentration | conc. | Temp | loading | Additive | Conversion | ee |
| Entry | Catalyst | [mol %] | [mol %] | [° C.] | [eq.] | ([mol %]) | [%] | [%] |
| A-1 | A-I | 0.5 | 0.18 | 0 | 3.5 | none | 99+ | 90 |
| A-2 | A-I | 1.0 | 0.10 | −20 | 3.5 | none | 99+ | ~93-94 |
| A-3 | A-I | 0.5 | 0.18 | 0 | 2.0 | none | 99+ | 88 |
| A-4 | A-I | 1.0 | 0.18 | 0 | 3.5 | benzoic | 99+ | 89 |
| | | | | | | acid (5) | | |
| A-5 | A-V | 1.0 | 0.18 | 0 | 3.5 | benzoic | 99+ | 78 |
| | | | | | | acid (5) | | |
| A-7 | A-VII | 1.0 | 0.18 | 0 | 3.5 | benzoic | 99+ | 90 |
| | | | | | | acid (5) | | |
| A-8 | A-IX | 1.0 | 0.18 | 0 | 3.5 | benzoic | 99+ | 89 |
| | | | | | | acid (5) | | |
| A-9 | A-III | 0.5 | 0.18 | 0 | 2.0 | benzoic | 99+ | 87 |
| | | | | | | acid (5) | | |
| A-9a | A-XI | 0.5 | 0.18 | 0 | 3.5 | benzoic | 99+ | 89 |
| | | | | | | acid (5) | | |
| A-9b | A-XIII | 0.5 | 0.18 | 0 | 3.5 | benzoic | 99+ | 88 |
| | | | | | | acid (5) | | |

In further samples, 300 mg myosmine and different catalysts at 0.5 mol % are dissolved with mol % benzoic acid in DCM as above, as specified in Table 2. The mixture is brought to a reaction temperature of 0° C. Trichlorosilane is added at 2.0 eq., and the mixture is reacted for the times given in Table 2. The reaction is quenched by adding an 8-30% sodium hydroxide solution (in water). The organic phase is separated and analyzed for enantiopurity by HPLC (mobile phase hexane:isopropanol=99:1 0.8 mL/min, stationary phase Daicel Chiralcel OD-H 25 cm×0.45 cm, detection wavelength 254 nm).

TABLE 2
Results of a series of examples with varying catalysts
and reaction times, following the procedure described
above. The enantiomeric excess refers to (S)-nornicotine.

| | | Reaction time | Conversion | ee |
|---|---|---|---|---|
| Entry | Catalyst | [hours] | [%] | [%] |
| A-10 | A-I | 2 | 86 | 87 |
| A-11 | A-I | 22 | 99+ | 87 |
| A-12 | A-VII | 2 | 80 | 89 |
| A-13 | A-VII | 22 | 99+ | 89 |
| A-14 | A-IX | 2 | 89 | 86 |
| A-15 | A-IX | 22 | 99 | 86 |

To test the influence of reaction time, loading amounts of myosmine and catalyst, solvent, additive, and silane load, varying amounts of myosmine and catalyst A-1 at varying amounts are dissolved with the respective additive in the respective solvent, as above, as specified in Table 3. The mixture is brought to a reaction temperature of 0° C. Trichlorosilane is added at the amount given, and the mixture is reacted for the times given in Table 3. The reaction is quenched by adding an 8-30% sodium hydroxide solution (in water). The organic phase is separated and analyzed for enantiopurity by HPLC (mobile phase hexane:isopropanol=99:1 0.8 mL/min, stationary phase Daicel Chiralcel OD-H 25 cm×0.45 cm, detection wavelength 254 nm).

TABLE 3
Results of a series of examples with varying catalysts, amounts of catalyst, temperature, silane loading equivalents,
and additive, following the procedure described above. The enantiomeric excess refers to (S)-nomicotine.

| | Myosmine | Myosmine | Catalyst | | Silane | | Reaction | | |
|---|---|---|---|---|---|---|---|---|---|
| | load | concentration | concentration | | loading | Additive | Eme | Conversion | ee |
| Entry | [mg] | (M) | [mol %] | Solvent | [eq.] | ([mol %]) | [hours] | [%] | [%] |
| A-16 | 300 | 0.18 | 0.5 | DCM | 2.0 | PTSA*H₂O (5) | 18 | 99 | 79 |
| A-17 | 300 | 0.18 | 0.5 | DCM | 2.0 | Malonic acid (5) | 18 | 99 | 74 |
| A-18 | 300 | 0.19 | 0.5 | DCM | 2.0 | Benzoic acid (10) | 18 | 99+ | 83 |
| A-19 | 500 | 0.50 | 0.5 | chlorobenzene | 3.5 | BHT (5) | 5 | 99 | 88 |
| A-20 | 100 | 0.18 | 0,5 | chloroform | 3.5 | none | 18 | 99 | 76 |
| A-21 | 100 | 0.18 | 0.5 | MeCN | 3.5 | none | 18 | 92 | 31 |
| A-22 | 500 | 0.5 | 0.5 | dichloromethane | 3.5 | Benzoic acid (2) | 3 | 95.5 | 89 |
| A-23 | 500 | 0.5 | 0.5 | chlorobenzene | 3.5 | Benzoic acid (2) | 3 | 99 | 89 |
| A-74 | 1000 | 1.0 | 1.0 | chlorobenzene | 3.5 | Benzoic acid (2) | 4 | 99 | 87 |
| A-25 | 1000 | 1.0 | 1.8 | anisole | 3.5 | Benzoic acid (2} | 4 | 99+ | 87 |
| A-26 | 1000 | 1.0 | 1.0 | toluene | 3.5 | Benzoic acid (2) | 4 | 59 | 86 |
| A-27 | 1000 | 1.0 | 2.5 | toluene | 3.5 | Benzoic acid (2) | 4 | 98 | 89 |
| A-28 | 1000 | 1.0 | 2.5 | p-xylene | 3.5 | Benzoic acid (2) | 4 | 33 | 81 |
| A-29 | 1000 | 1.0 | 2.5 | m-xylene | 3.5 | Benzoic acid (2) | 4 | 46 | 84 |
| A-30 | 1000 | 1.0 | 1.8 | toluene/chloroben | 3.5 | Benzoic acid (2) | 4 | 77 | 87 |
| | | | | zene (5/1) | | | | | |
| A-31 | 1000 | 1.0 | 1.0 | toluene/anisole | 3.5 | Benzoic acid (2) | 4 | 92 | 86 |
| | | | | (5/1) | | | | | |
| A-32 | 1000 | 1.0 | 1.0 | toluene/DCM (5/1) | 3.5 | Benzoic acid (2) | 4 | 99+ | 88 |
| A-33 | 20000 | 1.8 | 1.0 | Chlorobenzene | 3.5 | Benzoic acid (2) | 3 | 99+ | 88 |
| A-34 | 5000 | 1.0 | 1.0 | anisole | 3.5 | Benzoic acid (2) | 3 | 99+ | 86 |
| A-35 | 300 | 0.18 | 2.5 | DCM | 3.5 | none | 18 | 99+ | 89 |
| A-36 | 1000 | 0.49 | 0.5 | DCM | 3.5 | Benzoic acid (2) | 3 | 87 | 89 |
| A-37 | 1000 | 0.40 | 0.5 | DCM | 3.5 | Benzoic acid (3) | 3 | 92 | 87 |
| A-38 | 1000 | 0.49 | 0.5 | DCM | 2.1 | Benzoic acid (2) | 3 | 56 | 88 |
| A-39 | 1000 | 0.49 | 0.5 | DCM | 2.1 | Benzoic acid (4) | 3 | 66 | 86 |
| A-40 | 500 | 0.5 | 0.5 | chlorobenzene | 3.5 | Benzoic acid (2) | 3 | 99 | 88 |
| A-41 | 500 | 0.5 | 0.5 | chlorobenzene | 2.1 | Benzoic acid (2) | 3 | 99+ | 88 |
| A-42 | 500 | 0.5 | 1.0 | chlorobenzene | 2.1 | Benzoic acid (2) | 3 | 99+ | 86 |
| A-43 | 10000 | 1.0 | 0.4 | chlorobenzene | 1.5 | Benzoic acid (4) | 22 | 68 | 78 |
| A-44 | 10000 | 1.0 | 2.5 | chlorobenzene | 15 | Benzoic acid (4) | 22 | 99 | 85 |

PTSA = p-toluenesulfonic acid
BHT = 2,6-Di-tert-butyl-4-methylphenol

### Comparative Example 1: Myosmine Reduction without Catalyst

Myosmine (114 mg) is dissolved in 6.8 mL DCM. Trichlorosilane (370 mg) is added at 0° C. and the solution is stirred for 18 h. After the reaction time, 10% NaOH is added until all solids dissolve and the mixture is extracted twice with 40 mL DCM each. The organic phases are collected, dried, filtered, and reduced in vacuo, yielding racemic nornicotine (0% ee).

### Comparative Example 2: Myosmine Reduction with Catalyst C

1 eq myosmine (100 mg) and 10 mol % catalyst C are dissolved in 6.8 mL DCM as solvent. 2.0 equivalents (eq.) of trichlorosilane are added and the solution is stirred for 18 h. After the reaction time, 10% wt. % NaOH in water is added until all solids dissolved and the mixture is extracted twice with 40 mL DCM each. The organic phases are collected, dried, filtered and reduced in vacuo. The enantiomeric excess is determined by HPLC (mobile phase hexane:isopropanol=95:5 1 mL/min, stationary phase Daicel Chiralcel OD-H 25 cm×0.45 cm, detection wavelength 220 nm). While conversion was at 95%, no ee was obtained.

### Comparative Example 3: Myosmine Reduction with Catalyst D

1 eq myosmine (100 mg) and 33 mol % catalyst D are dissolved in 6.8 mL DCM as solvent. 3.5 equivalents (eq.) of trichlorosilane are added and the solution is stirred for 18 h. After the reaction time, 10% wt. % NaOH in water is added until all solids dissolved and the mixture is extracted twice with 40 mL DCM each. The organic phases are collected, dried, filtered and reduced in vacuo. The enantiomeric excess is determined by HPLC (mobile phase hexane:isopropanol=95:5 1 mL/min, stationary phase Daicel Chiralcel OD-H 25 cm×0.45 cm, detection wavelength 220 nm). While conversion was at 65%, no ee was obtained.

The entire disclosures of all applications, patents and publications, cited herein and of application No. PCT/EP2023/073883, filed 31 Aug. 2023, are incorporated by reference herein.

1. A method of producing nornicotine from myosmine, comprising:
reacting myosmine with at least one hydrosilane using a catalyst,
wherein the catalyst is a catalyst of Formula IIa-10: 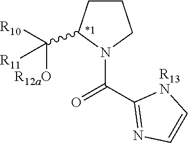
wherein in Formula IIa-10
R₁₀ and R₁₁ are the same or different and are chosen from H; F; Cl; Br; I; pseudohalogen groups;
linear alkyl groups with 1 to 10 C atoms that can optionally be substituted; branched alkyl groups with 3 to 10 C atoms that can optionally be substituted; aralkyl groups with 7 to 24 C atoms that can optionally be substituted; alkylaryl groups with 7 to 24 C atoms that can optionally be substituted; cycloalkyl groups with 3 to 20 C atoms that can optionally be substituted;
heterocycloalkyl groups with 1 to 15 C atoms that can optionally be substituted; aromatic and/or heteroaromatic groups with 2 to 40 C atoms that can optionally be substituted; alkyl heteroaromatic groups with 3 to 50 C atoms that can optionally be substituted; heteroaromatic groups with at least one alkyl residue with 3 to 50 C atoms that can optionally be substituted; alkyl cycloalkyl groups with 4 to 24 C atoms that can optionally be substituted; cycloalkyl alkyl groups with 4 to 24 C atoms that can optionally be substituted; alkyl heterocycloalkyl groups with 2 to 24 C atoms that can optionally be substituted; heterocycloalkyl alkyl groups with 2 to 24 C atoms that can optionally be substituted; carboxylic acid ester groups with 1 to 20 C atoms that can optionally be substituted; alkyl and/or (hetero)aryl oxycarbonyl groups with 1 to 20 C atoms that can optionally be substituted; and carboxamide groups with 1 to 20 C atoms that can optionally be substituted; wherein the optional substituents are chosen from at least one of F, Cl, Br, I, pseudohalogen groups, alkoxy groups with 1 to 20 C atoms, OH, NO₂ and NH₂;
R₁₂ₐ is chosen from H;
linear alkyl groups with 1 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups (including (CO)NH₂) with 1 to 20 C atoms that can optionally be substituted, alkoxy groups with 1 to 20 C atoms, OH, SH, NO₂ and NH₂;
linear alkenyl and/or alkynyl groups with 2 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
branched alkyl, alkenyl and/or alkynyl groups with 3 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
aralkyl groups with 7 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
alkylaryl groups with 7 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
cycloalkyl groups with 3 to 20 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
heterocycloalkyl groups with 1 to 15 C atoms that can optionally be substituted with at least one of F, Cl, Br, I, pseudohalogen groups, carboxylic acid ester groups with 1 to 20 C atoms that can optionally be substituted, OH, SH, NO₂ and NH₂, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
aromatic and/or heteroaromatic groups with 2 to 40 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
alkyl heteroaromatic groups with 2 to 50 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
heteroaromatic groups with at least one alkyl residue with 3 to 50 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
alkyl cycloalkyl groups with 4 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
cycloalkyl alkyl groups with 4 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
alkyl heterocycloalkyl groups with 2 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂;
heterocycloalkyl alkyl groups with 2 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO₂ and NH₂; SiR₂₃R₂₄R₂₅; wherein the optional substituents are chosen from at least one of F, Cl, Br, I, pseudohalogen groups, alkoxy groups with 1 to 20 C atoms, OH, SH, NO₂ and NH₂;
R₂₃, R₂₄, and R₂₅ are the same or different and are selected from H, linear alkyl groups with 1 to 4 C atoms that can optionally be substituted, branched alkyl group with 3 to 6 C atoms that can optionally be substituted, aromatic groups with 6 to 12 C atoms that can optionally be substituted, and aralkyl groups with 7 to 10 C atoms that can optionally be substituted, preferably wherein R₂₃, R₂₄, and R₂₅ are not all hydrogen at the same time and/or wherein R₂₃, R₂₄, and R₂₅ are not all methyl at the same time, further preferably wherein all of R₂₃, R₂₄, and R₂₅ are ethyl; wherein the optional substituents are chosen from at least one of F, Cl, Br, I, pseudohalogen groups, alkoxy groups with 1 to 20 C atoms, OH, NO₂ and NH₂; and
R₁₃ is chosen from H, linear alkyl groups with 1 to 10 C atoms that can optionally be substituted; branched alkyl groups with 3 to 10 C atoms that can optionally be substituted; aralkyl groups with 7 to 24 C atoms that can optionally be substituted; alkylaryl groups with 7 to 24 C atoms that can optionally be substituted; cycloalkyl groups with 3 to 20 C atoms that can optionally be substituted; heterocycloalkyl groups with 1 to 15 C atoms that can optionally be substituted;
aromatic and/or heteroaromatic groups with 2 to 40 C atoms that can optionally be substituted;
alkyl heteroaromatic groups with 3 to 50 C atoms that can optionally be substituted;
heteroaromatic groups with at least one alkyl residue with 3 to 50 C atoms that can optionally be substituted; alkyl cycloalkyl groups with 4 to 24 C atoms that can optionally be substituted;
cycloalkyl alkyl groups with 4 to 24 C atoms that can optionally be substituted; alkyl heterocycloalkyl groups with 2 to 24 C atoms that can optionally be substituted; heterocycloalkyl alkyl groups with 2 to 24 C atoms that can optionally be substituted; carboxylic acid ester groups with 1 to 20 C atoms that can optionally be substituted; alkyl and/or (hetero)aryl oxycarbonyl groups with 1 to 20 C atoms that can optionally be substituted; and carboxamide groups with 1 to 20 C atoms that can optionally be substituted; wherein the optional substituents are chosen from at least one of F, Cl, Br, I, pseudohalogen groups, alkoxy groups with 1 to 20 C atoms, OH, NO₂ and NH₂;
wherein the stereocenter *1 of the catalyst according to Formula IIa-10 is in (R) and/or (S) configuration.

2. The method according to claim 1, wherein
the reaction is conducted at a temperature in the range of −80° C. to 100° C., preferably −30 to 50° C., further preferably −20° C. to 25° C., and/or wherein
the reaction is conducted in a solvent comprising at least one non-protic solvent, preferably wherein the at least one solvent in selected from the group consisting of dichloromethane (DCM), (trichloromethyl)benzene, (trifluoromethyl)benzene, chloroform, o-xylene, m-xylene, p-xylene, toluene, benzene, chlorobenzene, ethers like anisole and Me-THF, acetonitrile, ethyl acetate (EtOAc), acetone and other ketones, and mixtures of two or more thereof.

3. The method according to claim 1, wherein the at least one hydrosilane has a Si—H bonding strength in the range of 84 to 100 kcal/mol, and/or wherein the at least one hydrosilane is selected from the group of silanes of formulas ABCSiH, ABSiH₂, and ASiH₃, wherein A, B and C can be the same or different and are selected from the group consisting of F; Cl; Br; I; alkyl groups with 1 to 20 C atoms that can optionally be substituted; aryl groups with 6 to 20 C atoms that can optionally be substituted; aralkyl groups with 7 to 24 C atoms that can optionally be substituted, alkoxy groups with 1 to 20 C atoms; and alkylaryl groups with 7 to 24 C atoms that can optionally be substituted, wherein the optional substituents are chosen from at least one of F, Cl, Br, I, pseudohalogen groups, and alkoxy groups with 1 to 20 C atoms; optionally wherein the at least one hydrosilane is Cl₃SiH.

4. The method according to claim 1, wherein the reaction is carried out in the presence of an acid and/or water and/or another protic solvent.

5. The method according to claim 1, wherein
the catalyst is present in an amount in the range of 0.001 to 60 mol %, preferably 0.005 to 55 mol %, e.g. 0.1 to 50 mol %, with respect to the amount of myosmine, optionally in the range of 0.3 to 20 mol % with respect to the amount of myosmine, and/or wherein
the hydrosilane is present in an amount in the range of 1 to 10 equivalents with respect to the amount of myosmine, optionally in the range of 1.2 to 8 equivalents, further optionally in the range of 1.5 to 6 equivalents, further optionally in the range of 2 to 5 equivalents with respect to the amount of myosmine.

6. The method according to claim 1, wherein the nornicotine is produced as a mixture of (R)- and (S)-nornicotine.

7. The method according to claim 1, further comprising removing the catalyst after reacting myosmine with at least one hydrosilane in the presence of the catalyst.

8. A composition, produced by the method according to claim 1, wherein the catalyst is not removed from the reaction mixture.

9. A composition, comprising
nornicotine, which is optionally a mixture of (R)- and (S)-nornicotine, and
a catalyst,
wherein the catalyst is a catalyst of Formula IIa-10;
wherein in Formula IIa-10
R10 and R11 are the same or different and are chosen from H; F; Cl; Br; I; pseudohalogen groups; linear alkyl groups with 1 to 10 C atoms that can optionally be substituted; branched alkyl groups with 3 to 10 C atoms that can optionally be substituted; aralkyl groups with 7 to 24 C atoms that can optionally be substituted; alkylaryl groups with 7 to 24 C atoms that can optionally be substituted; cycloalkyl groups with 3 to 20 C atoms that can optionally be substituted; heterocycloalkyl groups with 1 to 15 C atoms that can optionally be substituted;
aromatic and/or heteroaromatic groups with 2 to 40 C atoms that can optionally be substituted;
alkyl heteroaromatic groups with 3 to 50 C atoms that can optionally be substituted;
heteroaromatic groups with at least one alkyl residue with 3 to 50 C atoms that can optionally be substituted; alkyl cycloalkyl groups with 4 to 24 C atoms that can optionally be substituted;
cycloalkyl alkyl groups with 4 to 24 C atoms that can optionally be substituted; alkyl heterocycloalkyl groups with 2 to 24 C atoms that can optionally be substituted; heterocycloalkyl alkyl groups with 2 to 24 C atoms that can optionally be substituted; carboxylic acid ester groups with 1 to 20 C atoms that can optionally be substituted; alkyl and/or (hetero)aryl oxycarbonyl groups with 1 to 20 C atoms that can optionally be substituted; and carboxamide groups with 1 to 20 C atoms that can optionally be substituted; wherein the optional substituents are chosen from at least one of F, Cl, Br, I, pseudohalogen groups, alkoxy groups with 1 to 20 C atoms, OH, NO2 and NH2;
R12a is chosen from H;
linear alkyl groups with 1 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups (including (CO)NH2) with 1 to 20 C atoms that can optionally be substituted, alkoxy groups with 1 to 20 C atoms, OH, SH, NO2 and NH2;
linear alkenyl and/or alkynyl groups with 2 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
branched alkyl, alkenyl and/or alkynyl groups with 3 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
aralkyl groups with 7 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
alkylaryl groups with 7 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
cycloalkyl groups with 3 to 20 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
heterocycloalkyl groups with 1 to 15 C atoms that can optionally be substituted with at least one of F, Cl, Br, I, pseudohalogen groups, carboxylic acid ester groups with 1 to 20 C atoms that can optionally be substituted, OH, SH, NO2 and NH2, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and aromatic and/or heteroaromatic groups with 2 to 40 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
alkyl heteroaromatic groups with 2 to 50 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
heteroaromatic groups with at least one alkyl residue with 3 to 50 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
alkyl cycloalkyl groups with 4 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
cycloalkyl alkyl groups with 4 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
alkyl heterocycloalkyl groups with 2 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
heterocycloalkyl alkyl groups with 2 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2; SiR23R24R25; wherein the optional substituents are chosen from at least one of F, Cl, Br, I pseudohalogen groups, alkoxy groups with 1 to 20 C atoms, OH, SH, NO2 and NH2;
R23, R24, and R25 are the same or different and are selected from H, linear alkyl groups with 1 to 4 C atoms that can optionally be substituted, branched alkyl group with 3 to 6 C atoms that can optionally be substituted, aromatic groups with 6 to 12 C atoms that can optionally be substituted, and aralkyl groups with 7 to 10 C atoms that can optionally be substituted, preferably wherein R23, R24, and R25 are not all hydrogen at the same time and/or wherein R23, R24, and R25 are not all methyl at the same time, further preferably wherein all of R23, R24, and R25 are ethyl; wherein the optional substituents are chosen from at least one of F, Cl, Br, I pseudohalogen groups, alkoxy groups with 1 to 20 C atoms, OH, NO2 and NH2; and
R13 is chosen from H, linear alkyl groups with 1 to 10 C atoms that can optionally be substituted; branched alkyl groups with 3 to 10 C atoms that can optionally be substituted;
aralkyl groups with 7 to 24 C atoms that can optionally be substituted; alkylaryl groups with 7 to 24 C atoms that can optionally be substituted; cycloalkyl groups with 3 to 20 C atoms that can optionally be substituted; heterocycloalkyl groups with 1 to 15 C atoms that can optionally be substituted; aromatic and/or heteroaromatic groups with 2 to 40 C atoms that can optionally be substituted; alkyl heteroaromatic groups with 3 to 50 C atoms that can optionally be substituted; heteroaromatic groups with at least one alkyl residue with 3 to 50 C atoms that can optionally be substituted; alkyl cycloalkyl groups with 4 to 24 C atoms that can optionally be substituted; cycloalkyl alkyl groups with 4 to 24 C atoms that can optionally be substituted; alkyl heterocycloalkyl groups with 2 to 24 C atoms that can optionally be substituted; heterocycloalkyl alkyl groups with 2 to 24 C atoms that can optionally be substituted; carboxylic acid ester groups with 1 to 20 C atoms that can optionally be substituted; alkyl and/or (hetero)aryl oxycarbonyl groups with 1 to 20 C atoms that can optionally be substituted; and carboxamide groups with 1 to 20 C atoms that can optionally be substituted; wherein the optional substituents are chosen from at least one of F, Cl, Br, I, pseudohalogen groups, alkoxy groups with 1 to 20 C atoms, OH, NO2 and NH2;
wherein the stereocenter *1 of the catalyst according to Formula IIa-10 is in (R) and/or (S) configuration.

10. The composition according to claim 8, wherein the enantiomeric excess (ee) of one stereoisomer of nornicotine is in the range of 5 to 100%, optionally in the range of 10 to 99.9%, further optionally in the range of 20 to 99%.

11. A method of producing nicotine or a salt thereof from myosmine, comprising:
reacting myosmine with at least one hydrosilane in the presence of a catalyst to produce nornicotine according to claim 1, wherein the catalyst is of Formula IIa-10;
reacting the nornicotine to nicotine; and
optionally reacting the nicotine to a salt thereof.

12. A composition, produced by the method according to claim 11, wherein the catalyst is not separated from the reaction mixture.

13. A composition, comprising nicotine, which is optionally a mixture of (R)- and (S)-nicotine, or a salt thereof, and a catalyst, wherein the catalyst is a catalyst of Formula IIa-10:
wherein in Formula IIa-10
R10 and R11 are the same or different and are chosen from H; F; Cl; Br; I; pseudohalogen groups; linear alkyl groups with 1 to 10 C atoms that can optionally be substituted; branched alkyl groups with 3 to 10 C atoms that can optionally be substituted; aralkyl groups with 7 to 24 C atoms that can optionally be substituted; alkylaryl groups with 7 to 24 C atoms that can optionally be substituted; cycloalkyl groups with 3 to 20 C atoms that can optionally be substituted; heterocycloalkyl groups with 1 to 15 C atoms that can optionally be substituted;
aromatic and/or heteroaromatic groups with 2 to 40 C atoms that can optionally be substituted;
alkyl heteroaromatic groups with 3 to 50 C atoms that can optionally be substituted;
heteroaromatic groups with at least one alkyl residue with 3 to 50 C atoms that can optionally be substituted; alkyl cycloalkyl groups with 4 to 24 C atoms that can optionally be substituted;
cycloalkyl alkyl groups with 4 to 24 C atoms that can optionally be substituted; alkyl heterocycloalkyl groups with 2 to 24 C atoms that can optionally be substituted; heterocycloalkyl alkyl groups with 2 to 24 C atoms that can optionally be substituted; carboxylic acid ester groups with 1 to 20 C atoms that can optionally be substituted; alkyl and/or (hetero)aryl oxycarbonyl groups with 1 to 20 C atoms that can optionally be substituted; and carboxamide groups with 1 to 20 C atoms that can optionally be substituted; wherein the optional substituents are chosen from at least one of F, Cl, Br, I, pseudohalogen groups, alkoxy groups with 1 to 20 C atoms, OH, NO2 and NH2;
R12a is chosen from H;
linear alkyl groups with 1 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups (including (CO)NH2) with 1 to 20 C atoms that can optionally be substituted, alkoxy groups with 1 to 20 C atoms, OH, SH, NO2 and NH2;
linear alkenyl and/or alkynyl groups with 2 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
branched alkyl alkenyl and/or alkynyl groups with 3 to 10 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
aralkyl groups with 7 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
alkylaryl groups with 7 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
cycloalkyl groups with 3 to 20 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
heterocycloalkyl groups with 1 to 15 C atoms that can optionally be substituted with at least one of F, Cl, Br, I, pseudohalogen groups, carboxylic acid ester groups with 1 to 20 C atoms that can optionally be substituted, OH, SH, NO2 and NH2, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
aromatic and/or heteroaromatic groups with 2 to 40 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
alkyl heteroaromatic groups with 2 to 50 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
heteroaromatic groups with at least one alkyl residue with 3 to 50 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
alkyl cycloalkyl groups with 4 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
cycloalkyl alkyl groups with 4 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
alkyl heterocycloalkyl groups with 2 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2;
heterocycloalkyl alkyl groups with 2 to 24 C atoms that can optionally be substituted with at least one of F, Cl, Br, I, pseudohalogen groups, carboxylic acid ester groups with 1 to 20 C atoms that can optionally be substituted, alkyl and/or (hetero)aryl oxycarbonyl groups with 1 to 20 C atoms that can optionally be substituted, carboxamide groups with 1 to 20 C atoms that can optionally be substituted, alkoxy groups with 1 to 20 C atoms, OH, SH, NO2 and NH2; SiR23R24R25: wherein the optional substituents are chosen from at least one of F, Cl, Br, I pseudohalogen groups, alkoxy groups with 1 to 20 C atoms, OH, SH, NO2 and NH2;
R23, R24, and R25 are the same or different and are selected from H, linear alkyl groups with 1 to 4 C atoms that can optionally be substituted, branched alkyl group with 3 to 6 C atoms that can optionally be substituted, aromatic groups with 6 to 12 C atoms that can optionally be substituted, and aralkyl groups with 7 to 10 C atoms that can optionally be substituted, preferably wherein R23, R24, and R25 are not all hydrogen at the same time and/or wherein R23, R24, and R25 are not all methyl at the same time, further preferably wherein all of R23, R24, and R25 are ethyl; wherein the optional substituents are chosen from at least one of F, Cl, Br, I pseudohalogen groups, alkoxy groups with 1 to 20 C atoms, OH, NO2 and NH2; and
R13 is chosen from H, linear alkyl groups with 1 to 10 C atoms that can optionally be substituted; branched alkyl groups with 3 to 10 C atoms that can optionally be substituted;
aralkyl groups with 7 to 24 C atoms that can optionally be substituted; alkylaryl groups with 7 to 24 C atoms that can optionally be substituted; cycloalkyl groups with 3 to 20 C atoms that can optionally be substituted; heterocycloalkyl groups with 1 to 15 C atoms that can optionally be substituted; aromatic and/or heteroaromatic groups with 2 to 40 C atoms that can optionally be substituted; alkyl heteroaromatic groups with 3 to 50 C atoms that can optionally be substituted;
heteroaromatic groups with at least one alkyl residue with 3 to 50 C atoms that can optionally be substituted; alkyl cycloalkyl groups with 4 to 24 C atoms that can optionally be substituted;
cycloalkyl alkyl groups with 4 to 24 C atoms that can optionally be substituted; alkyl heterocycloalkyl groups with 2 to 24 C atoms that can optionally be substituted; heterocycloalkyl alkyl groups with 2 to 24 C atoms that can optionally be substituted; carboxylic acid ester groups with 1 to 20 C atoms that can optionally be substituted; alkyl and/or (hetero)aryl oxycarbonyl groups with 1 to 20 C atoms that can optionally be substituted; and carboxamide groups with 1 to 20 C atoms that can optionally be substituted; wherein the optional substituents are chosen from at least one of F, Cl, Br, I, pseudohalogen groups, alkoxy groups with 1 to 20 C atoms, OH, NO2 and NH2;
wherein the stereocenter *1 of the catalyst according to Formula IIa-10 is in (R) and/or (S) configuration.
